(19) United States
(12) Patent Application Publication
(10) Pub. No.: US 2013/0336905 A1
(43) Pub. Date: Dec. 19, 2013

(54) COMPOSITIONS OF ENHANCED VISCOSITY, CLARITY, OR BOTH ENHANCED VISCOSITY AND CLARITY

(71) Applicants: Hani M. Fares, Somerset, NJ (US); Christine M. Barrett, Oakland, NJ (US); Tracey Ross, Hewitt, NJ (US)

(72) Inventors: Hani M. Fares, Somerset, NJ (US); Christine M. Barrett, Oakland, NJ (US); Tracey Ross, Hewitt, NJ (US)

(73) Assignee: ISP Investments Inc., Wilmington (DE)

(21) Appl. No.: 13/642,030

(22) PCT Filed: Apr. 19, 2011
(86) PCT No.: PCT/US11/32993

§ 371 Date: Apr. 22, 2013

Related U.S. Application Data
(60) Provisional application No. 61/325,673, filed on Apr. 19, 2010.

(51) Int. Cl. A61K 8/81; A61Q 19/10; A61Q 19/00; A61Q 17/04; A61Q 19/08; A61Q 1/14; A61Q 15/00; A61Q 5/02; A61Q 1/10; A61Q 1/06; A61Q 1/04; A61Q 5/06; A61Q 5/12

(52) U.S. Cl. CPC A61K 8/8176; A61Q 5/06; A61K 8/8182; A61K 8/817; A61Q 19/10; A61Q 19/00; A61Q 17/04; A61Q 19/08; A61Q 5/12; A61Q 15/00; A61Q 5/02; A61Q 1/10; A61Q 1/06; A61Q 1/04; A61Q 1/14
USPC 424/59; 510/475; 510/130; 510/126; 424/70.7; 424/64; 510/136; 424/70.15; 424/65; 424/78.03; 510/513

(57) ABSTRACT

Provided herein are compositions that exhibit enhanced clarity, higher viscosity, or both enhanced clarity and higher viscosity for a wide range of application arts, such as personal care compositions. The compositions have lightly- to moderately-crosslinked PVP and at least one additive.

### FIELD OF THE INVENTION

The invention relates to compositions having lightly- to moderately crosslinked PVP, and at least one additive, and offer the formulation scientist enhanced clarity (i.e., reduced turbidity), higher viscosity, or both enhanced clarity and higher viscosity. These compositions lend themselves to a wide variety of compositions used in personal care or performance chemicals applications.

### DESCRIPTION OF RELATED ART

The invention is related to lightly- to moderately-crosslinked poly(N-vinyl-2-pyrrolidone). This polymer was first introduced in U.S. Pat. No. 5,073,614. The polymer is described as the precipitation polymerization product of N-vinyl-2-pyrrolidone monomer in an organic solvent, such as an aliphatic hydrocarbon solvent (particularly cyclohexane or heptane) or an aromatic hydrocarbon (such as toluene) in the presence of about 0.2% to 1% by weight of a crosslinking agent. The fine, white powders thus produced have an aqueous gel volume from about 15 mL to about 150 mL per gram of polymer, and a Brookfield viscosity in 5% aqueous solution of at least about 10,000 cP.

This lightly- to moderately-crosslinked poly(N-vinyl-2-pyrrolidone) (PVP) polymer also was the subject of U.S. Pat. No. 5,139,770. It provides examples wherein this polymer is incorporated into different types of personal care compositions.

Related is U.S. Pat. No. 5,716,634, which teaches a lightly-crosslinked N-vinyl lactam polymer in form of stable, clear, flowable, homogenized hydrogel, which may be used as a carrier for cosmetic/pharma active for hair or skin use. A controlled release drug-delivery composition comprising a lightly-crosslinked poly(N-vinyl-2-pyrrolidone) polymer is the subject of U.S. Pat. No. 5,252,611. Also, the production of lightly-crosslinked poly(N-vinyl-2-pyrrolidone) polymer in an oil-in-water or water-in-oil emulsion is taught in U.S. Pat. No. 6,177,068.

A summary of some properties of light- to moderately-crosslinked PVP is given in Shih, J. S., “Characteristics of lightly crosslinked poly(N-vinylpyrrolidone),” Polymer Materials: Science & Engineering Preprint, 72, 374, 1995.

Still more information on this lightly crosslinked PVP polymer is given in the following U.S. Pat. Nos. 5,162,417; 5,242,985; 5,268,117; 5,312,619; 5,470,884; 5,534,265; 5,614,583; 5,618,522; 5,622,168; 5,564,385; 5,645,859; 5,658,577; 5,663,258; 5,759,524; 5,843,881; 5,919,440; 5,968,528; 5,973,359; 5,997,887; 5,997,890; 6,001,377; 6,024,942; 6,174,533; 6,582,711; and 7,390,478. Related disclosure also is provided in U.S. patent applications 2003/0215413; 2007/0122501; and 2007/0154435. Also related are U.S. Statutory Registrations USH 2,013 and 2,043. Also related are German patents DE 69,533,239; 69,813,874; 69,814,066; 69,816,439; 69,818,037; 69,831,326; and 69,906,265. Related disclosure also is provided in European patent specification EP 777,465; and in PCT applications WO 1999/052501; 1999/052502; 2000/101523; 2000/048555; 2000/048568 and 2000/048569.

All of the above patents, patent applications, and Statutory Registrations, and the mentioned Shih article above are hereby incorporated in their entirety by reference.

Formulations of improved clarity and/or robust thickening ability are particularly useful and it would be desirable to provide compositions that exhibit one or both of these properties.

### SUMMARY OF THE INVENTION

Described herein are compositions having lightly- to moderately-crosslinked poly(N-vinyl-2-pyrrolidone) (PVP) that additionally contain one or more additive(s) that enhance composition clarity, enhance composition viscosity, or enhance both the clarity and viscosity. More specifically, it has been discovered that these additives reduce haze/turbidity and/or provide higher viscosity as measured by a Brookfield viscometer. Given these properties, compositions according to the invention offer substantial advantages in appearance and/or performance.

Also described is the use of these improved preparations in personal care and performance chemicals compositions.

### BRIEF DESCRIPTION OF THE DRAWINGS

FIG. 1 is a graph of viscosity as a function of addition level of Stabileze® QM for compositions produced in accordance with Example 3.

FIG. 2 is a graph of viscosity as a function of addition level of Stabileze® QM for compositions produced in accordance with Example 4.

FIG. 3 is a graph of viscosity as a function of addition level of RapiThix® A-100 for compositions produced in accordance with Example 5.

FIG. 4 is a graph of viscosity as a function of addition level of RapiThix® A-100 for compositions produced in accordance with Example 6.

FIG. 5 is a graph of viscosity as a function of addition level of PQ-37 for compositions produced in accordance with Example 7.

FIG. 6 is a graph of viscosity as a function of addition level of PQ-37 for compositions produced in accordance with Example 8.

FIG. 7 is a graph of viscosity as a function of addition level of lightly- to moderately-crosslinked PVP for compositions produced in accordance with Example 11.

FIG. 8 is a graph of turbidity as a function of addition level of lightly- to moderately-crosslinked PVP for compositions produced in accordance with Example 11.

FIG. 9 is a graph of turbidity as a function of addition level of surfactant for compositions produced in accordance with Example 13.

FIG. 10 is a graph of viscosity as a function of addition level of surfactant for compositions produced in accordance with Example 13.

### DETAILED DESCRIPTION

Due to the inherent complexity in these compositions, their ingredients, product forms, and uses, it will be appreciated that definitions of terms will help describe embodiments of the invention.

The term halogen refers to chloro, bromo, iodo and fluoro, and in particular bromo or chloro.

The terms “ultraviolet” and “UV” refer to electromagnetic radiation, especially solar electromagnetic radiation, with a wavelength from about 100 nm to about 400 nm, and includes the UV-A, UV-B, and UV-C subclassifications of such radiation.

The term “UV-A” refers to ultraviolet electromagnetic radiation with a wavelength from about 320 nm to about 400 nm, and includes UV-A1 (from about 340 nm to about 400 nm) and UV-A2 (from about 320 nm to about 340 nm).

The term “UV-B” refers to ultraviolet electromagnetic radiation with a wavelength from about 290 nm to about 320 nm.

The term “UV-C” refers to ultraviolet electromagnetic radiation with a wavelength from about 200 nm to about 290 nm.

The term “UV absorber” refers to compound that absorb, reflect, and/or scatter UV radiation.

The term personal care compositions (or formulations) refer to compositions intended for topical use on a mammal, such as man, horses, cats, and dogs. These compositions include skin, hair, scalp, foot, or lip compositions, including those compositions that can be purchased with and without a doctor's prescription. These personal care compositions find application on the hair for benefits such as: cleanliness, shine, vitality, body, fullness, split end mending, enhancing or changing color, partial/complete straightening, and partial/complete curling. Likewise, personal care compositions find application on the skin for benefits such as: moisturize, prevent wrinkles, treat wrinkles, wash, firm skin, treat blemishes, protect from ultraviolet radiation, protect from thermal damage, lighten skin color, remove dirt/soil/dead skin/blocked pores, and treat keratosis (e.g., corns, calluses, and warts). The personal care compositions also may comprise other active and non-active ingredients to assist in their benefit, delivery, spreadability, emolliency, film formation, stability, and/or thickening.

The term performance chemicals composition (or formulations) refers to non-personal care compositions that serve a broad variety of applications, and include non-limiting compositions such as: adhesives; agricultural, biocides, coatings, electronics, household-industrial-institutional (HI&I), inks, membranes, metal fluids, oilfield, paper, paints, plastics, printing, plasters, and wood-care compositions.

The term viscosity refers to the proportionality coefficient between shear stress and shear rate, and describes a composition's resistance to flow. Because viscosity is dependent on shear rate, specific measurement information (such as viscometer, flow apparatus/spindle, and shear rate) is required to properly define viscosity. As used herein, viscosity refers to the proportionality coefficient determined from low shear rate, rotational flow, especially the viscosity measured by the Brookfield LVT and Brookfield RVT viscometers typically operating at 10 revolutions per minute (rpm) at 25° C. References describing the Brookfield measurement of viscosities include the following, each of which is hereby incorporated in its entirety by reference: Thibodeau, L., “Measuring viscosity of pastes,” American Laboratory News, June 2004; McGregor, R. G., “Shelf life: does viscosity matter?” Pharmaceutical Online, Oct. 31, 2007; and McGregor, R. G., “When ointments disappoint, the viscosity story,” Brookfield Engineering brochure.

The term topical refers to any external parts of a mammal, such as man, horses, cats, and dogs, and especially man, and includes skin, hair, scalp, lips, and feet.

All percentages, ratio, and proportions used herein are based on a weight basis unless otherwise specified.

It has been discovered that some additives used in combination with lightly- to moderately-crosslinked PVP effectively increase blended composition clarity, increase composition viscosity or increase both composition clarity and viscosity. Typically, these additives comprise about 20% or less of the composition.

The following section describes the lightly- to moderately-crosslinked PVP, and then some embodiments of the invention are summarized.

### A Summary of Lightly- to Moderately-Crosslinked PVP

The term lightly- to moderately-crosslinked PVP, unless otherwise noted, specifically refers to polymer essentially consisting of lightly- to moderately-crosslinked poly(N-vinyl-2-pyrrolidone) having at least one of the following characteristics: (1) an aqueous swelling parameter defined by its gel volume from about 15 mL to about 300 mL per gram of polymer, more particularly from about 15 mL/g to about 250 mL/g, and yet more particularly from about 15 mL/g to about 150 mL/g, and (2) a Brookfield viscosity of 5% lightly- to moderately-crosslinked PVP in water at 25° C. of at least 2,000 cP, more particularly of at least about 5,000 cP, and yet more particularly of at least about 10,000 cP. Disclosure for these parameter ranges is provided in U.S. Pat. No. 5,073,614 and in Shih, J. S., et al. (1995). Synthesis methods for the lightly- to moderately-crosslinked PVP are disclosed in a number of documents, including U.S. Pat. Nos. 5,073,614; 5,654,385; and 6,177,068. It is appreciated by a polymer scientist skilled in the art that the method of synthesis is immaterial, inasmuch as the produced polymer achieves at least one of the abovedefined parameters. In certain tables and figures herein this polymer is called “lightly-crosslinked PVP” for text alignment. It is understood that it refers to lightly- to moderately-crosslinked PVP.

For example, U.S. Pat. No. '614 discloses different crosslinkers and crosslinker amounts that yield lightly- to moderately-crosslinked PVP suitable for use herein. The effects of crosslinker amount on swell volume and viscosity are graphically presented in Shih, J. S., et al. (“Characteristics of lightly crosslinked poly(N-vinylpyrrolidone),” Polymer Materials: Science & engineering Preprint, 72, 374, 1995). Thus, the lightly- to moderately-crosslinked PVP may be produced by the precipitation polymerization method of the '614 patent, by the hydrogel method described in the '385 patent, or by the non-aqueous, heterogeneous polymerization method of the '068 patent. Certainly, other techniques are contemplated to synthesize this polymer, provided the product meets the aqueous swelling parameter or Brookfield viscosity requirements set forth in the above paragraph. Final product viscosities may slightly vary for compositions containing lightly- to moderately-crosslinked PVP made by these different methods. Nonetheless, these variations are within the scope of the invention, as the lightly- to moderately-crosslinked PVPs thicken low pH compositions.

Unless otherwise specified, “lightly- to moderately-crosslinked PVP” does not refer to water-swellable but water-insoluble crosslinked PVP, such as the type sold into commercial trade under the trade name Polyclar by International Specialty Products, which differs from the lightly- to moderately-crosslinked PVP described above.

### EMBODIMENTS OF THE INVENTION

As mentioned earlier, it has been discovered that select additives can enhance the clarity, viscosity, or both the clarity and viscosity of compositions having the lightly- to moderately-crosslinked PVP described above. In a first embodiment, compositions are disclosed that comprise: (1) one or more additive(s) that enhance clarity and/or increase viscosity, and (2) lightly- to moderately-crosslinked PVP. A description of this first embodiment is described in the next paragraph.

In accordance with a second embodiment, compositions are disclosed consisting of: (A) at least one clarifying and/or viscosifying additive selected from a broader group of additives, (B) lightly- to moderately-crosslinked PVP, and (C) at least one solvent.

### First Embodiment

By a first embodiment, compositions are provided comprising (1) one or more additive(s) that enhance clarity and/or increase viscosity, and (2) lightly- to moderately-crosslinked PVP. Clarifying additives denoted in (1) include: cocomidopropyl betaine, decyl glucoside, disodium cocylglutamate, the copolymer of isobutylene, maleimide and hydroxyethylmaleimide (e.g., Aquaflex® SF-64, ISP), lauramidopropyl betaine, cocamide DEA, decyl glucoside, disodium laureth sulfosuccinate, potassium glycinate, polyquaternium-69 (e.g., Aquastyle™ 300), sodium laureth-2 sulfate, sodium laureth-3 sulfate, polyimide-1 (e.g., Aquaflex® XL-30, ISP), copolymers of N-vinyl-2-pyrrolidone (VP) and vinyl acetate (e.g., the PVP/VA series of polymers, ISP), copolymers of N-vinyl-2-caprolactam (VCL), VP, and dimethylaminoethyl methacrylate (DMAEMA) (e.g., Gaffix® VC-713 and Advantage® LC-A, ISP), copolymers of VP, VCL, and dimethylaminopropyl methacrylamide (DMAPA) (e.g., Aquaflex® SF-40, ISP), potassium lauryl sulfate, PVM/MA decadiene crosspolymer (e.g., Stabilize® QM, ISP), quaternium-26 (e.g., Ceraphyl® 65, ISP), sodium cocylglutamate, potassium cocylglycinate, polyquatemium-55 (e.g., Styleze® W-10 and W-20, ISP), and combinations thereof.

The addition level of the clarifying additive depends, in part, on the specifics of the formula. One skilled in the art understands how to make this determination, for example using the methods described in the Examples section. More generally, the amount of the clarifying additive ranges typically is at least 0.5% (w/w) of the composition, more particularly is at least 1% (w/w) of the composition, and yet more particularly is at least 5% (w/w) of the composition. The enhanced clarity, as represented by a reduction in the turbidity, in general is at least 100 nephelometric turbidity units (NTU) lower than a corresponding control without the clarifying additive, more particularly at least 200 NTU lower, and yet more particularly at least 400 NTU lower.

Viscosifying additives of this first embodiment denoted in (1) include: copolymers of VP, VCL, and DMAPA (e.g., Aquaflex® SF-40, ISP); copolymers of isobutylene, ethylmaleimide, and hydroxyethylmaleimide (e.g., Aquaflex® SF-64, ISP); polyquaternium-55 (e.g., Styleze® W-20, ISP), sodium laureth-2 sulfate, the copolymer of VP and DMAPA (e.g., Styleze® CC-10, ISP), and combinations thereof.

The addition level of the viscosifying additive depends, in part, on the specifics of the formula. One skilled in the art understands how to make this determination, for example using the methods described in the Examples section. More generally, the amount of the viscosifying additive is at least 0.5% (w/w) of the composition, more particularly is at least 1% (w/w) of the composition, and yet more particularly is at least 5% (w/w) of the composition. The enhanced viscosity, as represented by an increase in the viscosity, such as a Brookfield viscosity, in general is at least 900 cP higher than a corresponding control without the viscosifying additive, more particularly, 5,000 cP higher, and yet more particularly at least 10,000 cP higher. In accordance with certain embodiments, the increase in viscosity may be as high as 50,000 or even 100,000 cP.

### Second Embodiment

By a second embodiment, compositions are provided consisting of: (A) at least one clarifying and/or viscosifying additive selected from a broader group of additives, (B) lightly- to moderately-crosslinked PVP, and (C) at least one solvent. Clarifying additives included as (A) include those cited earlier as (1) as well as additional additives. For completeness, the clarifying additives (A) according to the second embodiment include: cocomidopropyl betaine, decyl glucoside, disodium cocylglutamate, the copolymer of isobutylene, maleimide and hydroxyethylmaleimide (e.g., Aquaflex® SF-64, ISP), lauramidopropyl betaine, potassium glycinate, polyquaternium-69 (e.g., Aquastyle™ 300), sodium laureth-2 sulfate, sodium laureth-3 sulfate, polyimide-1 (e.g., Aquaflex® XL-30, ISP), copolymers of VP and vinyl acetate (e.g., the PVP/VA series of polymers, ISP), cocamide DEA; copolymers of VCL, VP, and DMAEMA (e.g., Gaffix® VC-713 and Advantage® LC-A, ISP), copolymers of VP, VCL, and DMAPA (e.g., Aquaflex® SF-40, ISP), potassium lauryl sulfate, PVMIMA decadiene crosspolymer (e.g., Stabilize® QM, ISP), quaternium-26 (e.g., Ceraphyl® 65, ISP), sodium cocylglutamate, potassium cocylglycinate, polyquaternium-55 (e.g., Styleze® W-10 and W-20, ISP), polysorbate-20, ammonium lauryl sulfate, sodium alpha olefin sulfonate (i.e., compounds having the formula CₙH₂ₙ₋₁SO₃Na, wherein n=14-16 inclusive), ethanol, sorbitol, sodium lauryl sulfate, butylene glycol, hexylene glycol, copolymers of VP and dimethylaminoethylmethacrylate (Copolymer 958, ISP), poly(vinyl pyrrolidone) (PVP), polyquaternium-11 (e.g., Gafquat® 755N), polyquaternium-28 (e.g., Conditioneze® NT-20, ISP), propylene glycol, glycerin, phenethyl benzoate, and combinations thereof.

As described before, the addition level of the clarifying additive depends, in part, on the specifics of the formula. One skilled in the art understands how to make this determination, for example using the methods described in the Examples section. More generally, the amount of the clarifying additive is at least 0.5% (w/w) of the composition, more particularly is at least 1% (w/w) of the composition, and yet more particularly is at least 5% (w/w) of the composition. The enhanced clarity, as represented by a reduction in the turbidity, in general is at least 100 nephelometric turbidity units (NTU) lower than a corresponding control without the clarifying additive, more particularly at least 200 NTU lower, and yet more particularly at least 400 NTU lower.

Likewise, the viscosifying additives (B) of the second embodiment include those identified above as (2) as well as additional additives. For completeness, the viscosifying additives (B) according to the second embodiment include: copolymers of VP, VCL, and DMAPA (e.g., Aquaflex® SF-40, ISP); copolymers of isobutylene, ethylmaleimide, and hydroxyethylmaleimide (e.g., Aquaflex® SF-64, ISP); polyquaternium-55 (e.g., Styleze® W-20, ISP), copolymers of VP and DMAPA (e.g., Styleze® CC-10, ISP), polyquaternium-28 (e.g., Conditioneze® NT-20, ISP), ammonium lauryl sulfate, sodium lauryl sulfate, sodium laureth-2 sulfate, C12-C15 alkyl lactate (e.g., Ceraphyl® 41), and combinations thereof.

The addition level of the viscosifying additive depends, in part, on the specifics of the formula. One skilled in the art understands how to make this determination, for example using the methods described in the Examples section. More generally, the amount of the viscosifying additive is at least 0.5% (w/w) of the composition, more particularly is at least 1% (w/w) of the composition, and yet more particularly is at least 5% (w/w) of the composition. The enhanced viscosity, as represented by an increase in the viscosity, such as a Brookfield viscosity, in general is at least 900 cP higher than a corresponding control without the viscosifying additive, more particularly, 5000 cP higher, and yet more particularly at least 10,000 cP higher. In accordance with certain embodiments, the increase in viscosity may be as high as 50,000 or even 100,000 cP.

At least one solvent (C) is included in the second embodiment of the invention. Due to the wide scope of suitable solvents that may be used, the description of the at least one solvent (C) is described in its own section below.

Before proceeding to the solvent details, it is understood by the first and second embodiments that two or more clarifying additives to be used, or two or more viscosifying additives, or one or more clarifying additive with one or more viscosifying additive.

### Solvent(s)

The second embodiment of the invention provides compositions having (C) one or more solvent(s). Solvents summarized here may be regarded as an optional ingredient for compositions embraced by the first embodiment of the invention.

One approach for identifying one or more solvent(s) is the method by Shill (1995): One gram of the lightly- to moderately-crosslinked PVP is placed in a graduate cylinder and an excess of the solvent, e.g., 100 mL, is added, the cylinder is capped, and the contents are thoroughly mixed by inverting the cylinder 8-10 times. Then, the capped cylinder is allowed to stand at room temperature for 24 hours. A suitable solvent swells the polymer from about 15 mL/g to 300 mL/g. In one embodiment the solvent (or blend of solvents) provides a swell volume from about 15 mL/g to about 250 mL/g, and in another embodiment the solvent(s) create swell volume from about 15 mL/g to about 150 mL/g. Naturally, blends of solvents may be used, even solvents that do not meet the above criteria provided that at least one such solvent is used.

Compositions of the second embodiment may have any amount of solvent necessary to prepare the composition. Considerations on the amount of solvent to add can be based in part on the amount of the lightly- to moderately-crosslinked PVP, and the type(s) and amount(s) of additive(s), and the desired properties of the composition, such as its viscosity. In general, the amount of solvent is at least 40% (w/w), more particularly is at least 50% (w/w), and yet more particularly is at least 60% (w/w) of the composition. In accordance with certain embodiments, the solvent may be present in an amount of up to about 99%, more particularly about 95% and still more particularly about 90%

It is noted that some clarifying additives and viscosifying additives themselves are solvents for lightly- to moderately-crosslinked PVP. These additives automatically satisfy the requirement of the second embodiment for a solvent without the need for adding an additional solvent (which also is embraced by the invention). Such additives include, without limitation, ethanol, butylene glycol, hexylene glycol, glycerin, propylene glycol, and C12-C15 alkyl lactate.

Classes of solvents that satisfy these swell volume conditions are water, alcohols, esters, glycols, acids, hydrocarbon oils, non-hydrocarbon oils, and their various combinations. These gel solutions may have a pH that is acidic, neutral, or basic.

Examples of alcohols include: methanol, ethanol, 1-propanol, 2-propanol, 2-methoxypropanol, aminomethyl propanol, 1-butanol, 2-butanol, sec-butanol, 2-aminobutanol, 2-ethylbutanol, 2-methylbutanol, 3-methoxybutanol, behenyl alcohol, amyl alcohol, cetyl alcohol, cinnamyl alcohol, decyl alcohol, hexyl alcohol, cetearyl alcohol, isodecyl alcohol, lauryl alcohol, nonyl alcohol, oleyl alcohol, and myristyl alcohol.

Useful acids include, but are not limited to, solutions, dispersions, or emulsions of alpha and beta hydroxy acid, alpha hydroxyethanoic acid, alpha hydroxyoctanoic acid alpha hydroxycaprylic acid, ascorbic acid, adipic acid, citric acid, caprylic acid, capric acid, glycolic acid, lactic acid, lauric acid, malic acid, mixed fruit acids, myristic acid, palmitic acid, salicylic acid, stearic acid, tartaric acid, linoleic acid, linolenic acid, ricinoleic acid, oleic acid, elaidic acid, erucic acid, and combinations thereof.

Useful glycols include, but are not limited to: ethylene glycol, propylene glycol, butylene glycol, diethylene glycol, dipropylene glycol, hexylene glycol, hexaethylene glycol, polyethylene glycol, glycerin, and combinations thereof.

Examples of hydrocarbon oils are those that find use in personal care and performance chemicals compositions. Among these are petrolatum and mineral oil (i.e., paraffinic oils, naphthenic oils, and aromatic oils). Also suitable are the different vegetable oils (e.g., coconut, corn, cottonseed, olive, palm, peanut, rapeseed, Canola, safflower, sesame, soybean, sunflower, almond, cashew, hazelnut, macadamia, mongongo, pecan, pine nut, evening primrose, blackcurrant seed, borage seed, and grape seed). Also known are the essential oils from the berries, seeds, bark, wood, rhizome, leaves, resin, flowers, peel, or roots of plants (e.g., allspice, juniper, almond, anise, celery, cumin, nutmeg, cassia, cinnamon, sassafras, camphor, cedar, rosewood, sandalwood, aganvood, galangal, ginger, basil, bay leaf, common sage, eucalyptus, lemon grass, melaleuca, oregano, patchouli, peppermint, pine, rosemary, spearmint, tea tree, thyme, wintergreen, chamomile, clary sage, clove, geranium, hops, hyssop, jasmine, lavender, manuka, marjoram, orange, rose, ylang-ylang, bergamot, grapefruit, lemon, tangerine, and valerian). Essential oils are an approach for integrating an enhanced olfactory and/or tactile experience into the final composition.

Non-hydrocarbon oils also are known to those skilled in the art, and may be used with the invention. One class is the family of silicone oils, being oils based at least in part on silicon-oxygen linkages, and may be branched or unbranched.

The silicones may be present in the form of oils, waxes, resins, or gums. They may be volatile or non-volatile. The silicones can be selected from polyalkyl siloxanes, polyaryl siloxanes, polyalkyl aryl siloxanes, silicone gums and resins, and polyorgano siloxanes modified by organofunctional groups, and combinations thereof.

Suitable polyalkyl siloxanes include polydimethyl siloxanes with terminal trimethyl silyl groups or terminal dimethyl silanol groups (dimethiconol) and polyalkyl (C₁-C₂₀) siloxanes.

Suitable polyalkyl aryl siloxanes include polydimethyl methyl phenyl siloxanes and polydimethyl diphenyl siloxanes, linear or branched.

The silicone gums suitable for use herein include polydiorganosiloxanes particularly having a number-average molecular weight between 200,000 g/mol and 1,000,000 g/mol, used alone or mixed with a solvent. Examples include polymethyl siloxane, polydimethyl siloxane/methyl vinyl siloxane gums, polydimethyl siloxane/diphenyl siloxane, polydimethyl siloxane/phenyl methyl siloxane and polydimethyl siloxane/diphenyl siloxane/methyl vinyl siloxane.

Suitable silicone resins include silicones with a dimethyl/trimethyl siloxane structure and resins of the trimethyl siloxysilicate type.

The organo-modified silicones suitable for use in the invention include silicones such as those previously defined and containing one or more organofunctional groups attached by means of a hydrocarbon radical and grafted siliconated polymers. For example, the organo-modified silicone can be an amino-functional silicones.

Aminofunctional silicones represent another class of silicones that find application in this invention. Broadly speaking, these polymers contain at least one amine group and at least one silicon atom. These polymers represent a broad array of chemistries that may be ideal for creating the disclosed ultraviolet-absorbing compounds. For example, aminoalkylsiloxanes and aminoalkoxysiloxanes are but two examples of this polymer family, which can be further reacted to yield chemistries that include polyimides, polyureas, and polyurethanes.

Examples of aminofunetional silicones include isostearamidopropyl dimethylamine gluconate (and) propylene glycol amine-functional silicones; offered for commercial sale by The Lubrizol Corporation (Wickliffe, Ohio). Also available are a number of aminopropyl-terminated polydimethylsiloxanes, N-ethylamino-isobutyl terminated-polydimethyl siloxanes, aminopropylmethylsiloxane-dimethylsiloxane copolymers, aminoethyl-aminopropyl-methylsiloxane-dimethylsiloxane copolymers, aminoethyl-aminoisobutyl-methylsiloxane-dimethylsiloxane copolymers, and aminoethyl-aminopropylmethoxysiloxane-dimethylsiloxane copolymers, all of which are offered for commercial sale by Gelest, Inc. (Morrisville, Pa.). Blends of polymers having amine units also are contemplated.

The silicones may be used in the form of emulsions, nano-emulsions, or micro-emulsions.

Other alcohols, esters, glycols, acids, hydrocarbon oils, and non-hydrocarbon oils suitable for use in the personal care arts can be identified by one skilled in the art, for example, by referring to the infobase of the Personal Care Products Council and the Inventory and Common Nomenclature of Ingredients Employed in Cosmetic Products (dated 9 Feb. 2006), both of which are hereby incorporated herein their entirety by reference.

The pH of the solvent or even the final product may range from 1 to 14, as required by the final use. For example, acidic compositions of the invention can include skin care preparations (pH less than 2 to 5), shampoos (pH from about 5 to 7), and cleaners to remove mineral deposits (pH as low as 1).

Alkaline compositions also are known, including pH 8-9 for cleaning ferrous and non-ferrous metals, while hair relaxers, bleaches, and liquid drain cleaners typically have a pH of 13 or higher. Also included in this alkaline category are some paint removers/strippers and grease removers.

Also, the compositions of the invention can exhibit neutral or near neutral pH (from about 6 to about 8).

### Optional Formulary Ingredients

The invention fully encompasses compositions according to the first and second embodiments as summarized above. In each embodiment the compositions may be formulated with one or more optional ingredients as needed to create useful products. It was mentioned earlier that one or more solvent(s) is an optional ingredient for compositions according to the first embodiment, so that description will not be repeated here.

Surfactants suitable for use in the present invention include those selected from the anionic, cationic, amphoteric (also called zwitterionic), and non-ionic families of surfactants, and blends thereof.

Anionic surfactants include alkyl sulfate, alkyl ethoxylated sulfate, and combinations thereof, These materials have the respective formulas: (1) ROSO₃M and (2) RO(C₂H₄O)ₓSO₃M, wherein R is alkyl or alkenyl of from about 8 to about 30 carbon atoms, x is 1 to 10, and M is H or a salt-forming cation such as ammonium, alkanolamine containing C₁-C₃ alkyl groups such as triethanolamine, and monovalent and polyvalent metals such as the alkaline and alkaline earth metals. Preferred metals include sodium, potassium, magnesium, and calcium. The cation M of the anionic surfactant may be chosen such that the anionic surfactant component is water soluble. Solubility of anionic surfactants, in general, will depend upon the particular anionic surfactants and cations chosen. In one embodiment an anionic surfactant is soluble in the composition hereof.

For example, R has from about 10 to about 18 carbon atoms in both the alkyl and alkyl ethoxylated sulfates. The alkyl ethoxylated sulfates are typically made as condensation products of ethylene oxide and monohydric alcohols having from about 8 to about 24 carbon atoms. The alcohols can be derived from fats, e.g., coconut oil, palm kernel oil, or tallow, or can be synthetic. Such alcohols may be reacted with about 1 to about 10, more particularly from about 1 to about 4, and yet more particularly from about 2 to about 3.5 molar proportions of ethylene oxide and the resulting mixture of molecular species having, for example, an average of 3 moles of ethylene oxide per mole of alcohol, is sulfated and neutralized.

Specific examples of alkyl ether sulfates which may be used in the present invention are sodium and ammonium salts of coconut alkyl triethylene glycol ether sulfate; tallow alkyl triethylene glycol ether sulfate, and tallow alkyl hexaoxyethylene sulfate. For example, alkyl ether sulfates are those comprising a mixture of individual compounds, said mixture having an average alkyl chain length of from about 12 to about 16 carbon atoms and an average degree of ethoxylation of from about 1 to about 4 moles of ethylene oxide. The sulfate surfactant may be comprised of a combination of ethoxylated and nonethoxylated sulfates. Alkyl sulfates can provide excellent cleaning and lather performance. Alkyl ethoxylated sulfates can provide excellent cleaning performance.

Other suitable anionic detersive surfactants include, but are not limited to water-soluble salts of organic, sulfuric acid reaction products of the general formula R₁SO₃M where R₁ is selected from the group consisting of a straight or branched chain, saturated aliphatic hydrocarbon radical having from about 8 to about 24, particularly from about 10 to about 18, carbon atoms; and M is a cation such as ammonium, alkanolamines, such as triethanolamine, monovalent metals, such as sodium and potassium, and polyvalent metal cations, such as magnesium, and calcium. The cation M, of the anionic detersive surfactant may be chosen such that the detersive surfactant component is water soluble. Solubility will depend upon the particular anionic detersive surfactants and cations chosen. Examples of such detersive surfactants are the salts of an organic sulfuric acid reaction product of a hydrocarbon of the methane series, including iso neo and n-paraffins, having about 8 to about 24 carbon atoms, particularly from about 10 to about 18 carbon atoms and a sulfonating agent, e.g., SO₃, H₂SO₄, obtained according to known sulfonation methods, including bleaching and hydrolysis. The anionic detersive surfactant may be alkali metal and ammonium sulfonated C₁₀-C₁₈ n-paraffins.

Suitable classes of nonionic surfactants also include, but are not limited to:
1. The polyethylene oxide condensates of alkyl phenols, e.g., the condensation products of alkyl phenols having an alkyl group containing from about 6 to about 12 carbon atoms in either a straight chain or branched chain configuration, with ethylene oxide, the said ethylene oxide being present in amounts equal to from about 10 to about 60 moles of ethylene oxide per mole of alkyl phenol. The alkyl substituent in such compounds may be derived from polymerized propylene, diisobutylene, octane, or nonane, for example.
2. Those derived from the condensation of ethylene oxide with the product resulting from the reaction of propylene oxide and ethylene diamine products which may be varied in composition depending upon the balance between the hydrophobic and hydrophilic elements which is desired. For example, compounds containing from about 40% to about 80% polyoxyethylene by weight and having a molecular weight of from about 5,000 to about 11,000 resulting from the reaction of ethylene oxide groups with a hydrophobic base constituted of the reaction product of ethylene diamine and excess propylene oxide, said base having a molecular weight of the order of about 2,500 to about 3,000, are satisfactory.
3. The condensation product of aliphatic alcohols having from about 8 to about 18 carbon atoms, in either straight chain or branched chain configuration, with ethylene oxide, e.g., a coconut alcohol ethylene oxide condensate having from about 10 to about 30 moles of ethylene oxide per mole of coconut alcohol, the coconut alcohol fraction having from about 10 to about 14 carbon atoms.
4. Long chain tertiary amine oxides corresponding to the following general formula: R₁R₂R₃N→O, wherein R₁ contains an alkyl, alkenyl or monohydroxy alkyl radical of from about 8 to about 18 carbon atoms, from 0 to about 10 ethylene oxide moieties, and from 0 to about 1 glyceryl moiety, and R₂ and R₃ contain from about 1 to about 3 carbon atoms and from 0 to about 1 hydroxy group, e.g., methyl, ethyl, propyl, hydroxyethyl, or hydroxypropyl radicals. The arrow in the formula is a conventional representation of a semipolar bond. Non-limiting examples of amine oxides suitable for use in this invention include dimethyl-dodecylamine oxide, dimethyloctylamine oxide, dimethyl-decylamine oxide, dimethyl-tetradecylamine oxide, 3,6,9-tri-oxaheptadecyldiethylamine oxide, di(2-hydroxyethyl)-tetradecylamine oxide, 2-dodecoxyethyldimethylamine oxide, 3-dodecoxy-2-hydroxypropyldi (3-hydroxypropyeamine oxide, dimethylhexadecylamine oxide.
5. Long chain tertiary phosphine oxides corresponding to the following general formula: RR′R″P→O wherein R contains an alkyl, alkenyl or monohydroxyalkyl radical ranging from about 8 to about 18 carbon atoms in chain length, from 0 to about 10 ethylene oxide moieties and from 0 to about 1 glyceryl moiety and R′ and R″ are each alkyl or monohydroxyalkyl groups containing from about 1 to about 3 carbon atoms. The arrow in the formula is a conventional representation of a semipolar bond. Examples of suitable phosphine oxides include, but are not limited to: dodecyldimethylphosphine oxide, tetradecyldimethylphosphine oxide, tetradecylmethylethylphosphine oxide, 3,6,9,-trioxaoctadecyldimethylphosphine oxide, cetyidimethylphosphine oxide, 3-dodecoxy-2-hydroxypropyldi(2-hydroxyethyl) phosphine oxide, stearyldimethylphosphine oxide, cetylethylpropylphosphine oxide, oleyldiethylphosphine oxide, dodecyldiethylphosphine oxide, tetradecyldiethylphosphine oxide, dodecyldipropylphosphine oxide, dodecyldi(hydroxymethyl)phosphine oxide, dodecyldi(2-hydroxyethyl)phosphine oxide, tetradecylmethyl-2-hydroxypropylphosphine oxide, oleydimethylphosphine oxide, 2-liydroxydodecyldimethylphosphine oxide.
6. Long chain dialkyl sulfoxides containing one short chain alkyl or hydroxy alkyl radical of from about 1 to about 3 carbon atoms (usually methyl) and one long hydrophobic chain which include alkyl, alkenyl, hydroxy alkyl, or keto alkyl radicals containing from about 8 to about 20 carbon atoms, from 0 to about 10 ethylene oxide moieties and from 0 to about 1 glyceryl moiety. Examples include, but are not limited to: octadecyl methyl sulfoxide, 2-ketotridecyl methyl sulfoxide, 3,6,9,-trixaoctadecyl 2-hydroxyethyl sulfoxide, dodecyl methyl sulfoxide, oleyl 3-hydroxypropyl sulfoxide, tetradecyl methyl sulfoxide, 3-methoxytridecyl methyl sulfoxide, 3-hydroxytridecyl methyl sulfoxide, 3-hydroxy-4-dodecoxybutyl methyl sulfoxide.
7. Polyalkylene oxide modified dimethylpolysiloxanes, also known as dimethicone copolyols. These materials include the polyalkylene oxide modified dimethylpolysiloxanes of the following formulae:

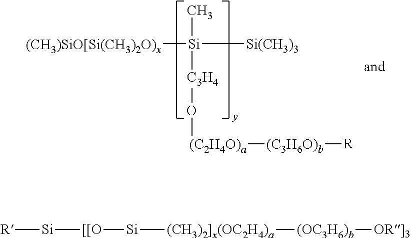

wherein R is hydrogen, an alkyl group having from 1 to about 12 carbon atoms, an alkoxy group having from 1 to about 6 carbon atoms or a hydroxyl group; R′ and R″ are alkyl groups having from 1 to about 12 carbon atoms; x is an integer of from 1 to 100, particularly from 20 to 30; y is an integer of 1 to 20, particularly from 2 to 10; and a and b are integers of from 0 to 50, particularly from 20 to 30. Dimethicone copolyols among those useful herein are disclosed in the following patent documents: U.S. Pat. No. 4,122,029; U.S. Pat. No. 4,265,878; and U.S. Pat. No. 4,421,769. Commercially available dimethicone copolyols, useful herein, include Silwet Surface Active Copolymers (manufactured by the Union Carbide Corporation); Dow Corning Silicone Surfactants (manufactured by the Dow Corning Corporation); Silicone Copolymer F-754 (manufactured by SWS Silicones Corp.); and Rhodorsil 70646 Fluid (manufactured by Rhone Poulenc, Inc.).

Anionic surfactants for use herein include: ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, and combinations thereof.

Surfactant systems useful in the present application also may comprise cationic surfactants. Cationic surfactants typically contain amino or quaternary ammonium hydrophilic moieties which are positively charged when dissolved in the aqueous composition of the present invention. Cationic surfactants among those useful herein are disclosed in the following documents: McCutcheon's, Detergents & Emulsifiers, (M.C. Publishing Co., North American edition 1989); Schwartz, et al., Surface Active Agents, Their Chemistry and Technology. New York: Interscience Publishers, 1949; U.S. Pat. Nos. 3,155,591; 3,929,678; 3,959,461; and 4,387,090.

Among the quaternary ammonium-containing cationic surfactant materials useful herein are those of the general formula:

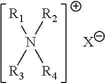

wherein R₁-R₄ are independently an aliphatic group of from about 1 to about 22 carbon atoms, or an aromatic, alkoxy, polyoxyalkylene, alkylamido, hydroxyalkyl, aryl or alkylaryl group having from about 12 to about 22 carbon atoms; and X is an anion selected from halogen, acetate, phosphate, nitrate and alkylsulfate radicals. The aliphatic groups may contain, in addition to carbon and hydrogen atoms, ether linkages, and other groups such as amino groups.

Other quaternary ammonium salts useful herein have the formula:

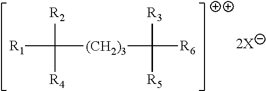

wherein R₁ is an aliphatic group having from about 16 to about 22 carbon atoms, R₂, R₃, R₄, R₅, and R₆ are selected from hydrogen and alkyl having from about 1 to about 4 carbon atoms, and X is an ion selected from halogen, acetate, phosphate, nitrate and alkyl sulfate radicals. Such quaternary ammonium salts include tallow propane diammonium dichloride.

Quaternary ammonium salts include monoalkyltrimethylammonium chlorides and dialkyldimethylammonium chlorides and trialkyl methyl ammonium chlorides, wherein at least one of the alkyl groups have from about 12 to about 22 carbon atoms and are derived from long-chain fatty acids, such as hydrogenated tallow fatty acid (tallow fatty acids yield quaternary compounds wherein the long chain alkyl groups are predominately from 16 to 18 carbon atoms). Examples of quaternary ammonium salts useful in the present invention include, but are not limited to, stearyl trimethyl ammonium chloride, ditallowedimethyl ammonium chloride, ditallowedimethyl ammonium methyl sulfate, dihexadecyl dimethyl ammonium chloride, di(hydrogenated tallow) dimethyl ammonium chloride, dioctadecyl dimethyl ammonium chloride, dieicosyl dimethyl ammonium chloride, didocosyl dimethyl ammonium chloride, di(hydrogenated tallow) dimethyl ammonium acetate, dihexadecyl dimethyl ammonium chloride, dihexadecyl dimethyl ammonium acetate, ditallow dipropyl ammonium phosphate, ditallow dimethyl ammonium nitrate, di(coconutalkyl) dimethyl ammonium chloride, and stearyl dimethyl benzyl ammonium chloride, ditallow dimethyl ammonium chloride, dicetyl dimethyl ammonium chloride, stearyl dimethyl benzyl ammonium chloride and cetyl trimethyl ammonium chloride are examples of quaternary ammonium salts useful herein.

In addition to the anionic and cationic surfactants described above, amphoteric surfactant components useful in the disclosed compositions include those known to be useful in personal cleansing compositions. Examples of amphoteric surfactants suitable for use in the composition herein are described in U.S. Pat. No. 5,104,646 (Bolich Jr., et al.) and U.S. Pat. No. 5,106,609 (Bolich Jr., et al.). Examples of amphoteric detersive surfactants which can be used in the compositions of the present invention are those which are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate.

Other amphoterics, sometimes classified as zwitterionics, such as betaines can also be used in the present invention. Such zwitterionics are considered as amphoterics in the present invention where the zwitterionic has an attached group that is anionic at the pH of the composition. Examples of betaines useful herein include the high alkyl betaines, such as. The sulfobetaines may be represented by coco dimethyl sulfopropyl betaine, stearyl dimethyl sulfopropyl betaine, lauryl dimethyl sulfoethyl betaine, lauryl bis-(2-hydroxyethyl) sulfopropyl betaine and the like; amidobetaines and amidosulfobetaines, wherein the RCONH(CH₂)₃ radical is attached to the nitrogen atom of the betaine are also useful in this invention.

Specifically, examples of amphoteric surfactants for use in the invention include: coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl)carboxymethyl betaine, stearyl bis-(2-hydroxypropyl)carboxymethyl betaine, oleyl dimethyl gamma-carboxypropyl betaine, and lauryl bis-(2-hydroxypropyl)-α-carboxyethyl betaine. Other examples of amphoteric surfactants are sodium 3-dodecyl-aminopropionate, sodium 3-dodecylaminopropane sulfonate, sodium lauroamphoacetate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and the products sold under the trade name Miranol™ and described in U.S. Pat. No. 2,528,378.

Other surfactants may be used in the surfactant system of the present invention, that the surfactant is also chemically and physically compatible with the essential components of the present invention, or does not otherwise unduly impair product performance, aesthetics or stability.

In one embodiment the surfactants that serve as clarity-enhancing additives are selected from the group consisting of sodium lauryl sulfate, sodium laureth sulfate, potassium lauryl sulfate, sodium cocoyl glutamate, potassium cocoylglycinate, and lauramidopropyl betaine. Sodium lauryl sulfate and sodium laureth sulfate are two examples of viscosity-enhancing additives useful for the present invention.

Alcohols are another class of optional ingredients to be included in the invention's compositions. In another aspect of the invention, enhanced viscosity and/or clarity of the gel solution is attained when an alcohol is added. As used herein, the term alcohol refers to any molecule having at least one hydroxyl (—OH) functional group. These alcohols may exist in the liquid or solid state. There are several classifications of alcohols that find utility in the invention, each of which is considered separately.

In one aspect the alcohol can be a simple alcohol. For example, ethanol at a 10% (w/w) addition level was found to increase the viscosity of gel solution by +900 cP. Other simple alcohols are contemplated to function as additives of the invention, including: denatured ethanol, methanol, 1-propanol, 2-propanol, 2-methoxypropanol, aminomethyl propanol, 1-butanol, 2-butanol, sec-butanol, 2-aminobutanol, 2-ethylbutanol, 2-methylbutanol, 3-methoxybutanol, behenyl alcohol, amyl alcohol, cetyl alcohol, cinnamyl alcohol, decyl alcohol, hexyl alcohol, cetearyl alcohol, isodecyl alcohol, lauryl alcohol, nonyl alcohol, oleyl alcohol, and myristyl alcohol.

Effective alcohols also were found from the polyol subfamily of alcohols, which are those alcohols have more than one hydroxyl functional group. Polyols that reduce the turbidity/haze of the gel solutions, and/or that increase the gel solution viscosity include: propylene glycol, glycerin, butylene glycol, hexylene glycol, and sorbitol. Blends of these polyols with other alcohols also are contemplated.

Polymers represent yet another category of optional ingredients. In some embodiments the polymers may have one or more N-vinyl lactam monomers, such as N-vinyl-2-pyrrolidone (VP) or N-vinyl-2-caprolactam (VCL). Many N-vinyl lactam polymers are known, and representatives of this group include: poly(VP-co-dimethylaminoethyl methacrylate), poly(VP-co-vinyl acetate), poly(VP-co-styrene), poly(VP-co-dimethylaminopropyl methacrylamide), poly(VP-co-acrylic acid), poly(VCL-ter-VP-ter-dimethylaminoethyl methacrylate), poly(VCL-ter-VP-ter-dimethylaminopropyl methacrylamide), poly(VP-ter-lauryl methacrylate-ter-acrylic acid), and the quaternized polymers: poly(VP-co-dimethylaminoethyl methacrylate), poly(VP-co-methacrylamido propyltrimethyl ammonium chloride), poly(VP-ter-dimethylaminopropyl methacrylate-ter-methacrylamido propyltrimethyl ammonium chloride), and poly(VCL-ter-dimethylaminopropyl methacrylamide-ter-hydroxyethyl methacrylate).

### Preparations Incorporating the Invention's Compositions

Given their enhanced clarity and/or viscosity, a wide variety of preparations may be created that incorporate the invention's compositions to serve the personal care and performance chemicals arts.

For example, the in accordance with some aspects, the compositions exhibit improved clarity (lower haze or turbidity), accordingly lending themselves to aesthetic or functional preparations. Similarly, the compositions disclosed herein may demonstrate higher viscosity, which can be advantageously utilized in thicker preparations and/or simpler formulations wherein viscosifier(s) level(s) are reduced or eliminated.

Many consumers exhibit a preference personal care preparations of reduced whiteness, haze, and/or opacity, even if only for shelf-appeal, Favored are transparent or almost transparent preparations. Personal care compositions in this category include products for the hair, skin, nails, and lips, such as body washes, skin lotions, hair conditioners, hair rinses, hair shampoos, hair styling agents, sunscreen, tanning products, hair sprays, make-up removers, and moisturizers. Where clarity is of lesser importance, the boosted viscosity provided by the invention's compositions can facilitate thickening of the final product, e.g., to reduce cost or enhance sensory qualities like texture, smoothness, consistency, and feel.

The personal care and performance chemicals preparations can contain any effective addition level of the invention's compositions. An effective amount depends on the starting formulation, the end use, and the type and amount of clarifying and/or viscosifying additive. That is, “an effect amount” is relative to the specifics of the art field. For example, an “effect amount” for an ultra-hold hair gel may be more than for a shampoo, lotion, or rinse wash. A skilled formulation scientist can determine the appropriate amount. In general, the compositions disclosed herein may find application at addition levels of at least 2% (w/w), more particularly at least 10% (w/w), and yet more particularly at least 30% (w/w) of the total formulation weight.

In one embodiment, the enhanced personal care composition is an anti-perspirant, a deodorant, or a combination anti-perspirant/deodorant product. The invention provides for these compositions having a clearer or less-hazy appearance, a thicker consistency (for example, appropriate for roll-on, liquid gels, or sticks), or both a clearer/less-hazy appearance and a thicker consistency. Additional disclosure for anti-perspirants, deodorants, and anti-perspirant/deodorants is provided in international application WO2010/105030, the contents of which are incorporated herein their entirety by reference.

In another embodiment, the invention provides for sunscreen products, which are those products having one or more UV absorbers. Sunscreen formulations include beach and non-beach products that are applied to the face, décolleté, lips, and skin to treat and/or protect against erythema, burns, wrinkles, lentigo (“liver spots”), skin cancers, keratotic lesions, and cellular changes of the skin; and to hair to treat and/or protect against color changes, lack of luster, tangles, split ends, unmanageability, and embrittlement. Other marketed names for this product segment include sun blocks, tanning products, sun absorbers, all-day protection, and baby sun care. These compositions include at least one organic or inorganic UV absorber, and combinations thereof may be used, e.g., for compositions that protect a wide range of UV-A and UV-B wavelengths. A first aspect of this embodiment provides for aqueous, alcoholic, hydroalcoholic, and non-aqueous sunscreen products having enhanced clarity. In a second aspect this embodiment provides for aqueous, alcoholic, hydroalcoholic, and non-aqueous sunscreen products having enhanced viscosity.

Examples of UV absorbers include: octyl salicylate (2-ethylhexyl salicylate, Escalol® 587); pentyl dimethyl PABA; octyl dimethyl PABA (padimate 0, Escalol® 507); benzophenone-1; benzophenone-6 (Uvinul® D-49); 2-(2H-benzotriazole-2-yl)-4,6-di-tert-pentylphenol (Uvinul® 3028); ethyl-2-cyano-3,3-diphenylacrylate (Uvinul® 3035); homomethyl salicylate (homosalate); bis-ethylhexyloxyphenol methoxyphenyl triazine (bemotrizinol, Tinosorb® S); methyl-(1,2,2,6,6-pentamethyl-4-piperidyl)-sebacate (Uvinul® 4092H); benzenepropanoic acid, 3,5-bis(1,1-dimethyl-ethyl)-4-hydroxy-, C7-C9 branched alkyl esters (Irganox® 1135); 2-(2H-benzotriazole-2-yl)-4-methylphenol (Uvinul® 3033P); diethylhexyl butamido triazone (iscotrizinol); amyl dimethyl PABA (lisadimate, glyceryl PABA); 4,6-bis(octylthiomethyl)-o-cresol (Irganox® 1520); CAS number 65447-77-0 (Uvinul® 5062H, Uvinul® 5062GR); red petroleum; ethylhexyl triazone (Uvinul® T-150); octocrylene (Escalol® 597); isoamyl-p-methoxycinnamate (amiloxate, Neo Heliopan® E1000); drometrizole; titanium dioxide; 2,4-di-tert-butyl-6-(5-chloro-2H-benzotriazole-2-yl)-phenol (Uvinul® 3027); 2-hydroxy-4-octyloxybenzophenone (Uvinul® 3008); benzophenone-2 (Uvinul® D-50); diisopropyl methylcinnamate; PEG-25 PABA; 2-(1,1-dimethylethyl)-6-[[3-(1,1-demethylethyl)-2-hydroxy-5-methylphenyl]methyl-4-methylphenyl acrylate (Irganox® 3052); drometrizole trisiloxane (Mexoryl® XL); menthyl anthranilate (meradimate); bis-(1,2,2,6,6-pentamethyl-4-piperidyl)-sebacate; butyl methoxydibenzoylmethane (avobenzone, Escalol® 517); 2-ethoxyethyl p-methoxycinnamate (cinnoxate); benzylidene camphor sulfonic acid (Mexoryl® SL); dimethoxyphenyl-[1-(3,4)]-4,4-dimethyl 1,3-pentanedione; zinc oxide; N,N′-hexane-1,6-diyl-bis[3-(3,5-di-tert-butyl-4-hydroxyphenylpropionamide)] (Irganox® 1098); pentaerythritol tetrakis[3-(3,5-di-tert-butyl-4-hydroxyphenyl)propionate] (Irganox® 1010); 2,6-di-tert-butyl-4-[4,6-bis(octylthio)-1,3,5-triaziN-2-ylamino]phenol (Irganox® 565); 2-(2H-benzotriazole-2-yl)-4,6-bis(1-methyl-1-phenylethyl)phenol (Uvinul® 3034); trolamine salicylate (triethanolamine salicylate); diethylanolamine p-methoxycinnamate (DEA methoxycinnamate); polysilicone-15 (Parsol® SLX); CAS number 152261-33-1 (Uvinul® 5050H); 4-methylbenzylidene camphor (Eusolex® 6300, Parsol® 5000); bisoctrizole (Tinosorb® M); benzenamine, N-phenyl-, reaction products with 2,4,4-trimethylpentene (Irganox® 50507); sulisobenzone, Escalor 577); (2-ethylhexyl)-2-cyano-3,3-diphenylacrylate (Uvinul® 3039); digalloyl trioleate; polyacrylamido methylbenzylidene camphor; glyceryl ethylhexanoate dimethoxycinnamate; 1,3-bis-[(2′-cyano-3′,3′-diphenylacryloyl)oxy]-2,2-bis-{[(2]-cyano-; bis-(2,2,6,6-tetramethyl-4-piperidyl)-sebacate (Uvinul® 4077H); benzophenone-5; 1,3,5-tris(3,5-di-tert-butyl-4-hydroxybenzyl)-1,3,5-triazine-2,4,6(1H,3H,5H)-trione (Irganox® 3114); hexamethylendiamine (Uvinul® 4050H); benzophenone-8 (dioxybenzone); ethyl-4-bis(hydroxypropyl)aminobenzoate (roxadimate); 6-tert-butyl-2-(5-chloro-2H-benzotriazole-2-yl)-4-methylphenol (Uvinul® 3026); p-aminobenzoic acid (PABA); 3,3,3″,5,5′,5″-hexa-tert-butyl-α-α′-α″-(mesitylene-2,4,6-triyl)tri-p-cresol (Irganox® 1130); lawsone with dihydroxyacetone; benzophenone-9 (Uvinul® DS-49); benzophenone-4; ethylhexyl dimethoxy benzylidene dioxoimidazoline propionate; N,N′-bisformyl-N,N′-bis-(2,2,6,6-tetramethyl-4-piperidinyl)-; 3-benzylidene camphor (Mexoryl® SD); terephthalylidene dicamphor sulfonic acid; camphor benzalkonium methosulfate (Mexoryl® SO); bisdisulizole disodium (Neo Heliopan® AP); etocrylene; ferulic acid; 2-(2H-benzotriazole-2-yl)-4-(1,1,3,3-tetramethylbutyl)-phenol (Uvinul® 3029); 4,6-bis(dodecylthiomethyl)-o-cresol (Irganox® 1726); beta-2-glucopyranoxy propyl hydroxy benzophenone; phenylbenzimidazole sulfonic acid (ensulizole, Eusolex® 232, Parsol® HS); benzophenone-3 (oxybenzone, Escalol® 567); diethylamine hydroxybenzoyl hexylbenzoate (Uvinul® A Plus); 3′,3′-diphenylacryloyl)oxy]methyl}-propane (Uvinul® 3030); and ethylhexyl p-methoxycinnamate (Escalol® 557).

It is recognized that the availability of UV absorbers in sun-care compositions often depends on local regulatory laws; hence, the above list may include UV absorbers that are not allowed in certain regions.

In particular, one or more UV absorber may be selected from the following: p-aminobenzoic acid (PABA), Padimate O, ensulizole, cinoxate, benzophenone-3, enzophenone-8, homosalate, meradimate, octocrylene, 2-ethylhexyl-p-methoxycinnamate, octyl salicylate, sulisobenzone, trolamine salicylate, avobenzone, ecamsule, titanium dioxide, zinc oxide, 4-methylbenzylidene, Tinosorb® M, neo heliopan AP, mexoryl XL, benzophenone-9, Uvinul® T150, Uvinul® A Plus, Uvasorb® HEB, Parsol® SLX, and isopentenyl-4-methoxycinnamate.

Additional disclosure of this embodiment is provided in U.S. provisional application 61/447,751, the contents of which are incorporated in its entirety by reference.

In yet another embodiment, the invention provides for skin care compositions having acidic pH, i.e., less than 7. These compositions include skin, hair, scalp, foot, or lip compositions, including those compositions that can be purchased with and without a doctor's prescription. These personal care compositions can provide any number of known benefits, such as: moisturize, prevent wrinkles, treat wrinkles, firm skin, treat blemishes, protect from ultraviolet radiation, protect from thermal damage, lighten skin color, remove dirt/soil/dead skin/blocked pores, and treat keratosis (e.g., corns, calluses, and warts). As with other embodiments, the invention's enhanced clarity may be useful to create compositions that are less noticeable after applying and drying. The active agent may be selected from hydroxy acids (e.g., alpha, beta, alpha-beta, and polyhydroxy variants), vitamin C serum, citric acid, salicylic acid, glycolic acid, tartaric acid, lactic acid, and combinations of these agents.

Alpha hydroxy acids can exhibit high epidermis penetration so that they may exert a maximum effect on the underlying dermis layer. Thus, the most effective alpha hydroxy acids are those of small molecular weight, such as glycolic acid and lactic acid. This preference, however, is not to say that the invention does not work in thickening higher molecular weight acids. Rather, this preference merely recognizes a special class of hydroxy acids that are used in many personal care and pharmaceutical compositions.

Like their alpha counterparts, beta hydroxy acids also find utility in the invention and in skin care products due to their ability to penetrate the epidermis and activity in the dermal layer. Beta hydroxy acids are those molecules having a carboxylic acid group and a hydroxyl group separated by two carbon atoms. Again, both naturally occurring and synthetic beta hydroxy acids are known and may be used in the invention's compositions. Specific examples of beta hydroxy acids include, but are not limited to: beta hydroxybutanoic acid, tropic acid, trethocanic acid, salicylic acid, and 5-(n-octanoyl) salicylic acid.

Also for use in the thickened topical compositions are alpha-beta hydroxy acids. As the same suggests, these acids contain at least one alpha hydroxy acid group and one beta hydroxy acid group. Examples of alpha beta hydroxy acids include: malic acid, citric acid, and tartaric acid.

A final member of the hydroxy acid family is the polyhydroxy acid, which, as the name suggests, are molecules having at least one carboxylic acid functional group and more than 1 hydroxyl group. Polyhydroxy acids also may be naturally occurring or synthetically manufactured, and have a higher molecular weight than glycolic acid or lactic acid. As a result, polyhydroxy acids are less penetrating than these two alpha hydroxy acids, and, as a result, provide gentler skin effects, typically with reduced irritation. Examples of suitable polyhydroxy acids include lactobionic acid, galactose, and gluconic acid.

Examples of performance chemicals compositions served by the invention include: coatings, adhesives, inks paints, biocides, pesticides, insecticides, antimicrobials, cleaning, disinfectants, sanitary compositions.

The invention now will be illustrated by the following non-limiting examples that highlight the compositions and methods described herein.

### EXAMPLES

### Example 1

### Clarifying Additives

Compositions according to the invention were made comprising 5% lightly- to moderately-crosslinked PVP (w/w finished product) in deionized water with the clarifying additives of Table 1. Clarity was measured as turbidity using a calibrated Hach 2100P Turbidimeter.

All additives enhanced composition clarity (i.e., reduced turbidity) relative to the control preparation, which comprised lightly- to moderately crosslinked PVP in water (Table 1). Reductions in turbidity ranged from 127 NTU for glycerin to 975 NTU for potassium lauryl phosphate.

TABLE 1
Clarity-enhancing additives of Example 1

| | addition level | turbidity | Δ turbidity |
|---|---|---|---|
| additive | (% w/w) | (NTU) | (NTU) |
| none (control) | | 1000 | |
| butylene glycol | 10.0 | 728 | −272 |
| cocamide DEA | 10.0 | 28.2 | −971.8 |
| decyl glucoside | 10.0 | 102 | −898 |
| disodium laureth sulfosuccinate | 10.0 | 823 | −177 |
| glycerin | 10.0 | 883 | −127 |
| hexylene glycol | 10.0 | 430 | −570 |
| lauramidopropyl betaine | 10.0 | 327 | −673 |
| potassium cocoylglycinate | 10.0 | 758 | −242 |
| potassium lauryl phosphate | 10.0 | 25 | −975 |
| propylene glycol | 10.0 | 788 | −212 |
| sodium cocoyl glutamate | 10.0 | 240 | −760 |
| sodium laureth sulfate | 10.0 | 357 | −643 |
| sodium lauryl sulfate | 5.0 | 100 | −900 |
| sorbitol | 10.0 | 509 | −491 |
| copolymer of VCL, VP, & | 10.0 | 373 | −627 |
| DMAEMA (Gaffix ® VC-713) | | | |
| quaternium-26 | 10.0 | 170 | −830 |
| VP/DMAEMA copolymer | 5.0 | 532 | −468 |
| (Copolymer 958, ISP) | | | |

### Example 2

### Viscosifying Additives

Compositions according to the invention were made comprising 5% lightly- to moderately-crosslinked PVP (w/w finished product) in deionized water with the additives named in Table 2. Solution viscosity was measured using a Brookfield RVT viscometer with spindle 6 at 10 rpm.

All additives enhanced viscosity relative to the control preparation, which comprised lightly- to moderately crosslinked PVP in water. Substantial increases in viscosity were measured, ranging from a 900 cP increase for ethanol up to 139,900 cP increase for sodium lauryl sulfate. These results indicate these additives are useful to boost the viscosity of compositions comprising lightly-crosslinked PVP.

TABLE 2
Viscosity-enhancing additives of Example 2

| | addition level | viscosity | Δ viscosity |
|---|---|---|---|
| additive | (% w/w) | (cP) | (cP) |
| none (control) | | 29,100 | |
| butylene glycol | 10.0 | 34,100 | +5,000 |
| ethanol | 10.0 | 30,000 | +900 |
| hexylene glycol | 10.0 | 37,600 | +37,600 |
| potassium cocylglycinate | 10.0 | 34,500 | +5,400 |
| propylene glycol | 10.0 | 40,000 | +10,900 |
| quaternium-26 | 10.0 | 55,700 | +26,600 |
| sodium cocylglutamate | 10.0 | 38,700 | +9,600 |
| sodium laureth sulfate | 10.0 | 42,200 | +13,100 |
| sodium lauryl sulfate | 5.0 | 169,000^{†} | +139,900 |

^{†}Measured with spindle 7.

### Comparative Example 1

### Materials that do not Enhance Viscosity

Compositions were prepared having 5% lightly- to moderately-crosslinked PVP (w/w finished product) in deionized water with the four materials and addition levels of Table 3. Solution viscosity was measured using a Brookfield RVT viscometer with spindle 6 at 10 rpm.

These materials at these addition levels are not considered to be viscosity enhancers for compositions comprising lightly- to moderately crosslinked PVP.

TABLE 3
Materials of Comparative Example 1

| | addition level | viscosity | Δ viscosity |
|---|---|---|---|
| material | (% w/w) | (cP) | (cP) |
| none (control) | | 29,100 | |
| potassium cocoylglycinate | 10.0 | 9,100 | −20,000 |
| sodium cocoyl glutamate | 10.0 | 7,980 | −21,120 |
| lauryamidopropyl betaine | 10.0 | 210 | −28,890 |
| potassium lauryl phosphate | 10.0 | 90 | −29,010 |

### Comparative Example 2

### Materials that do not Enhance the Clarity

Compositions were prepared having 5% lightly- to moderately-crosslinked PVP (w/w finished product) in deionized water with the materials included in Table 4. Solution viscosity was measured using a Brookfield RVT viscometer with spindle 6 at 10 rpm.

These materials at these addition levels are not considered to be viscosity enhancers for compositions comprising lightly- to moderately-crosslinked PVP, as reduced viscosities were measured.

TABLE 4
Materials of Comparative Example 2

| | addition level | viscosity | Δ viscosity |
|---|---|---|---|
| material | (% w/w) | (cP) | (cP) |
| none (control) | | 29,100 | |
| glycerin | 10.0 | 27,500 | −1,600 |
| sorbitol | 10.0 | 24,900 | −4,200 |
| Copolymer 958 | 5.0 | 18,400 | −10,700 |
| Advantage ® LC-A | 10.0 | 17,500 | −11,600 |

### Example 3

### Viscosity Synergy of PVM/MA Decadiene Crosspolymer in 60% Ethanol/40% Water

Five formulations of the invention were prepared having varying amounts of lightly-to moderately-crosslinked PVP and PVM/MA decadiene crosspolymer (Stabileze® QM, ISP) in a quantity-sufficient (q.s.) blend of 60% ethanol/40% water (w/w) (Table 5). The neutralizer 2-amino-2-methyl-1-propanol (AMP-95, Angus Chemie GmbH) was added to formulas having Stabilize® QM. The amount of total polymer was maintained constant at 1.5% (w/w). Five control formulas also were made with up to 1.5% (w/w) Stabileze® QM but no lightly- to moderately-crosslinked PVP. An additional set of control formulas contained up to 1.5% (w/w) lightly- to moderately crosslinked PVP in the same ethanol/water blend, but no Stabileze® QM and no AMP-95. Viscosity was measured at room temperature (about 25° C.) using a Brookfield viscosity with spindle T-D operating at 10 rpm.

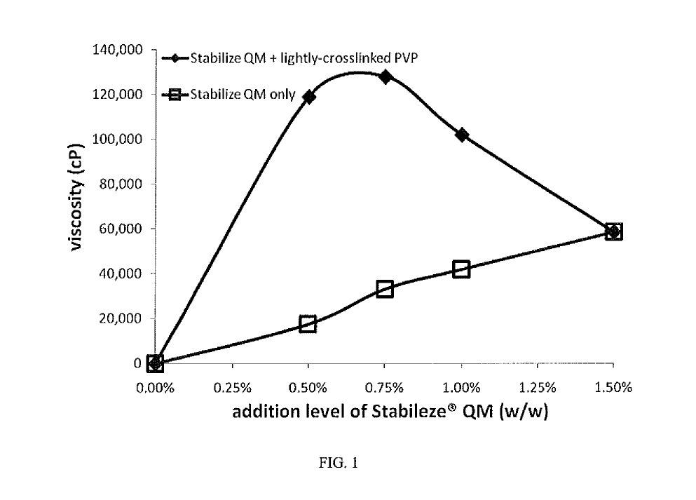

The combination of lightly- to moderately-crosslinked PVP with Stabileze® QM exhibited a viscosity synergy. The control formulas having up to 1.5% of only lightly- to moderately-erosslinked PVP attained a viscosity of less than 200 cP. Adding Stabileze® QM increased viscosity up to 128,000 cP (FIG. 1). This increase was not due to Stabileze® QM alone, the viscosity of which never exceeded 60,000 eP.

TABLE 5
Formulations of Example 3

| | | addition level to make 100 g batch (g) | | | | |
|---|---|---|---|---|---|---|
| | | lightly- | | | | |
| | | crosslinked PVP | Stabileze ® QM | AMP-95 | 60% EtOH/ | viscosity |
| formula | | (100% active) | (100% active) | (50% active) | 40% water | (cP) |
| of the | 1 | 0.00 | 1.50 | 3.50 | q.s. | 58,600 |
| invention | 2 | 0.50 | 1.00 | 2.34 | q.s. | 102,000 |
| | 3 | 0.75 | 0.75 | 1.75 | q.s. | 128,000 |
| | 4 | 1.00 | 0.50 | 1.16 | q.s. | 119,000 |
| | 5 | 1.50 | 0.00 | 0.00 | q.s. | <200 |
| control | 1 | 0 | 1.50 | 3.5 | q.s. | 58,600 |
| | 2 | 0 | 1.00 | 2.34 | q.s. | 42,000 |
| | 3 | 0 | 0.75 | 1.75 | q.s. | 33,200 |
| | 4 | 0 | 0.50 | 1.16 | q.s. | 17,600 |
| | 5 | 0 | 0.00 | 0.00 | q.s. | <200 |
| | | up to 1.5% | 0 | 0 | 0 | <200 |

### Example 4

### Viscosity Synergy of PVM/MA Decadiene Crosspolymer in Water

Example 3 was substantially repeated using water instead of the ethanol/water blend.

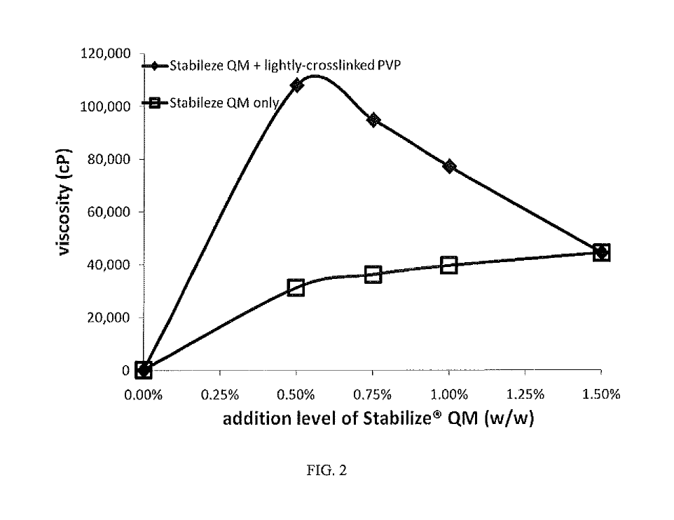

A viscosity synergy was noted for these polymers in water, with a maximum viscosity of 108,000 cP (Table 6, FIG. 2). Again, this viscosity increase was not due to Stabilize® QM alone, which showed a maximum viscosity of 44,400 cP.

TABLE 6
Formulations of Example 4

| | | addition level to make 100 g batch (w/w) (g) | | | | |
|---|---|---|---|---|---|---|
| | | lightly- | Stabileze ® | AMP-95 | | |
| | | crosslinked PVP | QM (100% | (50% | | viscosity |
| formula | | (100% active) | active) | active) | water | (cP) |
| of the | 1 | 0.00 | 1.50 | 3.50 | q.s. | 44,400 |
| invention | 2 | 0.50 | 1.00 | 2.34 | q.s. | 77,200 |
| | 3 | 0.75 | 0.75 | 1.75 | q.s. | 94,800 |
| | 4 | 1.00 | 0.50 | 1.16 | q.s. | 108,000 |
| | 5 | 1.50 | 0.00 | 0.00 | q.s. | <200 |
| control | 1 | 0 | 1.50 | 3.5 | q.s. | 44,400 |
| | 2 | 0 | 1.00 | 2.34 | q.s. | 39,600 |
| | 3 | 0 | 0.75 | 1.75 | q.s. | 36,200 |
| | 4 | 0 | 0.50 | 1.16 | q.s. | 31,200 |
| | 5 | 0 | 0.00 | 0.00 | q.s. | <200 |
| | | up to 1.5% | 0 | 0 | 0 | <200 |

### Example 5

### Viscosity Synergy of Sodium Polyacrylate in 75% Ethanol, 25% Water

Example 6 was substantially repeated using sodium polyacrylate (RapiThix® A-100) instead of Stabileze® QM, and using a blend of 75% ethanol/25% water (w/w). There was no addition of 2-amino-2-methyl-1-propanol in these formulas.

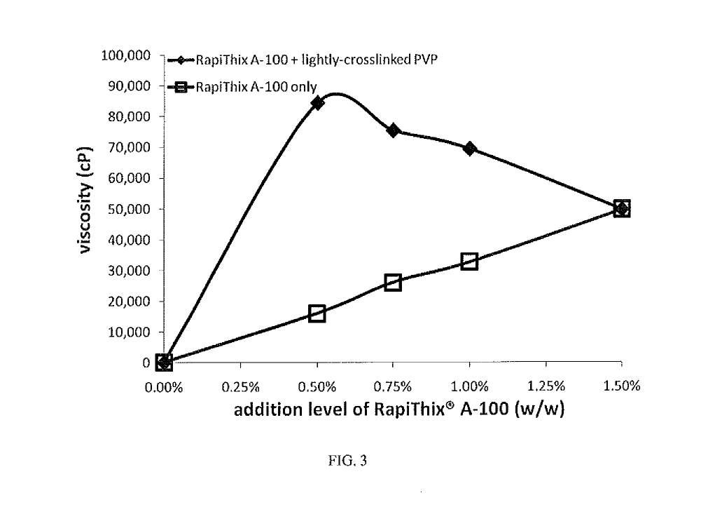

A viscosity synergy was found for these polymers in water, with a maximum viscosity of 84,400 cP (Table 7, FIG. 3). Again, this viscosity increase was not due to RapiThix® A-100 alone, which showed a maximum viscosity of 49,600 cP.

TABLE 7
Formulations of Example 5

| | | addition level to make 100 g batch (g) | | | |
|---|---|---|---|---|---|
| | | | RapiThix ® | | |
| | | | A-100 | | |
| | | lightly-crosslinked | (100% | 75% ethanol/ | viscosity |
| formula | | PVP (100% active) | active) | 25% water | (cP) |
| of the | 1 | 0.00 | 1.50 | q.s. | 49,600 |
| invention | 2 | 0.50 | 1.00 | q.s. | 69,400 |
| | 3 | 0.75 | 0.75 | q.s. | 75,400 |
| | 4 | 1.00 | 0.50 | q.s. | 84,400 |
| | 5 | 1.50 | 0.00 | q.s. | <200 |
| control | 1 | 0 | 1.50 | q.s. | 49,600 |
| | 2 | 0 | 1.00 | q.s. | 32,600 |
| | 3 | 0 | 0.75 | q.s. | 26,000 |
| | 4 | 0 | 0.50 | q.s. | 16,000 |
| | 5 | 0 | 0.00 | q.s. | <200 |
| | | up to 1.5% | 0 | 0 | <200 |

### Example 6

### Viscosity Synergy of Sodium Polyacrylate in Water

Example 5 was substantially repeated using water instead of the ethanol/water blend. As in Example 8 there was no 2-amino-2-methyl-1-propanol in these formulas.

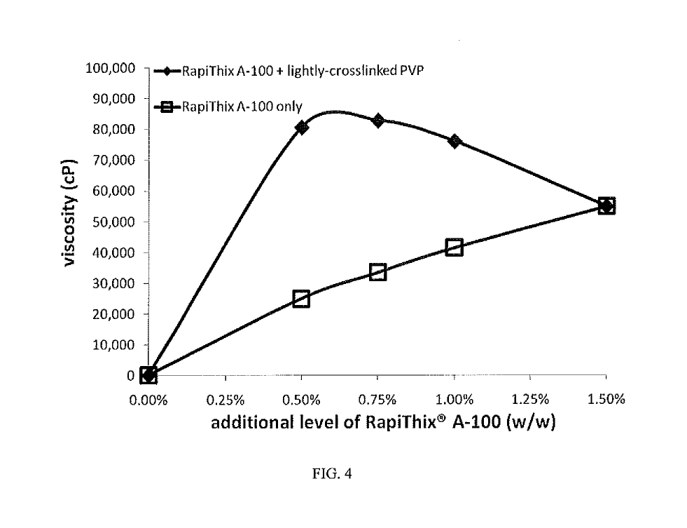

A viscosity synergy was noted for these polymers in water, with a maximum viscosity of 82,800 cP (Table 8, FIG. 4). Again, this viscosity increase was not due to RapiThix® A-100 alone, which showed a maximum viscosity of 54,800 cP.

TABLE 8
Formulations of Example 6

| | | addition level to make 100 g batch (g) | | | |
|---|---|---|---|---|---|
| | | lightly-crosslinked | RapiThix ® A-100 | | viscosity |
| formula | | PVP (100% active) | (100% active) | water | (cP) |
| of the | 1 | 0.00 | 1.50 | q.s. | 54,800 |
| invention | 2 | 0.50 | 1.00 | q.s. | 76,000 |
| | 3 | 0.75 | 0.75 | q.s. | 82,800 |
| | 4 | 1.00 | 0.50 | q.s. | 80,600 |
| | 5 | 1.50 | 0.00 | q.s. | <200 |
| control | 1 | 0 | 1.50 | q.s. | 54,800 |
| | 2 | 0 | 1.00 | q.s. | 41,400 |
| | 3 | 0 | 0.75 | q.s. | 33,400 |
| | 4 | 0 | 0.50 | q.s. | 25,000 |
| | 5 | 0 | 0.00 | q.s. | <200 |
| | | up to 1.5% | 0 | 0 | <200 |

### Example 7

### Viscosity Synergy of PQ-37 in 60% Ethanol, 40% Water

Example 3 was substantially repeated using polyquaternium-37 (PQ-37) instead of Stabileze® QM. The example retained the 60% ethanol/40% water (w/w) solvent blend, but 2-amino-2-methyl-1-propanol was not added.

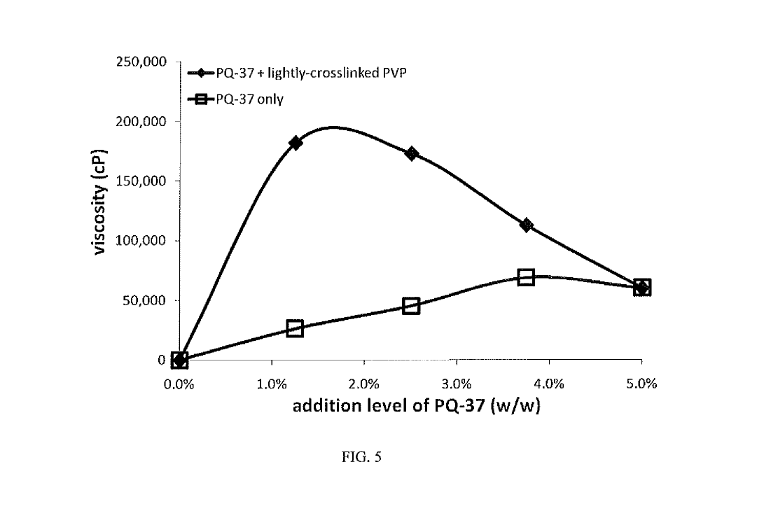

A viscosity synergy was noted for these polymers in water, with a maximum viscosity of 182,000 cP (Table 9, FIG. 5). This viscosity increase was not due to PQ-37 alone, which showed a maximum viscosity of 69,000 cP.

TABLE 9
Formulations of Example 7

| | | addition level to make 100 g batch (g) | | | |
|---|---|---|---|---|---|
| | | lightly-crosslinked | PQ-37 | 60% ethanol/ | viscosity |
| formula | | PVP (100% active) | (50% active) | 40% water | (cP) |
| of the | 1 | 0.00 | 10.00 | q.s. | 60,200 |
| invention | 2 | 1.25 | 7.50 | q.s. | 113,000 |
| | 3 | 2.50 | 5.00 | q.s. | 173,000 |
| | 4 | 3.75 | 2.50 | q.s. | 182,000 |
| | 5 | 5.00 | 0.00 | q.s. | 23,400 |
| control | 1 | 0 | 10.00 | q.s. | 60,200 |
| | 2 | 0 | 7.50 | q.s. | 69,000 |
| | 3 | 0 | 5.00 | q.s. | 45,400 |
| | 4 | 0 | 2.50 | q.s. | 26,200 |
| | 5 | 0 | 0.00 | q.s. | <200 |
| | | up to 1.5% | 0 | 0 | <200 |

### Example 8

### Viscosity Synergy of PQ-37 in 60% Ethanol, 40% Water

Example 7 was substantially repeated using water as the solvent.

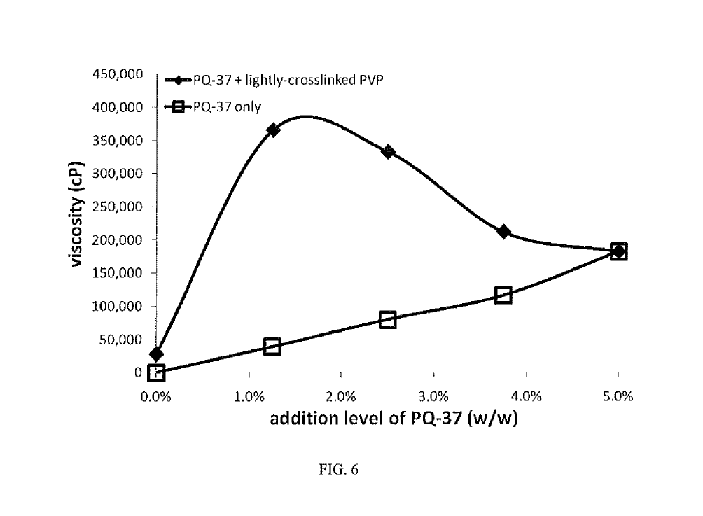

A viscosity synergy was noted for these polymers in water, with a maximum viscosity of 366,000 cP (Table 10, FIG. 6). This viscosity increase was not due to PQ-37 alone, which showed a maximum viscosity of 182,000 cP.

TABLE 10
Formulations of Example 8

| | | addition level to make 100 g batch (g) | | | |
|---|---|---|---|---|---|
| | | lightly-crosslinked | PQ-37 | 60% ethanol/ | viscosity |
| formula | | PVP (100% active) | (50% active) | 40% water | (cP) |
| of the | 1 | 0.00 | 10.00 | q.s. | 182,000 |
| invention | 2 | 1.25 | 7.50 | q.s. | 212,000 |
| | 3 | 2.50 | 5.00 | q.s. | 333,000 |
| | 4 | 3.75 | 2.50 | q.s. | 366,000 |
| | 5 | 5.00 | 0.00 | q.s. | 28,000 |
| control | 1 | 0 | 10.00 | q.s. | 182,000 |
| | 2 | 0 | 7.50 | q.s. | 116,000 |
| | 3 | 0 | 5.00 | q.s. | 79,800 |
| | 4 | 0 | 2.50 | q.s. | 38,800 |
| | 5 | 0 | 0.00 | q.s. | <200 |
| | | up to 1.5% | 0 | 0 | <200 |

### Example 9

### Effect of Ethanol on Clarity and Viscosity

A composition were prepared having 5% lightly- to moderately-crosslinked PVP and 95% solvent, where the solvent system had varying ratios of ethanol and water (Table 11). The turbidity and viscosities were measured as reported in Examples 1 and 2.

The turbidity decreased as the solvent system shifted to higher amounts of ethanol (lower amounts of water). Concurrently, the viscosity increased.

TABLE 10
The effects of ethanol on turbidity and viscosity

| solvent ratio | | turbidity | viscosity |
|---|---|---|---|
| ethanol | water | (NTU) | (cP) |
| 0 | 100 | 1,000 | 29,100 |
| 10 | 90 | 1,000 | 30,000 |
| 25 | 75 | 517 | 57,600 |
| 50 | 50 | 320 | 62,500 |
| 75 | 25 | 229 | 74,800 |
| 100 | 0 | 219 | 83,600 |

### Example 10

### Effect of Various Styling Polymers on Clarity and Viscosity

Aqueous compositions were made having 5% lightly- to moderately crosslinked PVP and 10% (w/w) of various hair styling polymers. Again, turbidity and viscosity were measured as reported in Examples 1 and 2.

Eight of the hair styling polymers improved the composition clarity, with as much as a 627 NTU drop in turbidity produced with 10% (w/w) Advantage LC-A (Table 11A). Four of the hair styling polymers increased viscosity and are representative of viscosifying additives of the invention (Table 11B). The increases in viscosity ranged from +7,700 cP to +23,200 cP. Without being bound by theory, it appears that polyquats perform favorably as clarifying and viscosifying additives for lightly- to moderately-crosslinked PVP, and compositions and uses thereof are contemplated.

TABLE 11A
The effects of various hair styling polymers on clarity

| | | turbidity | Δturbidity |
|---|---|---|---|
| hair styling polymer | trade name | (NTU) | (NTU) |
| none | | 1,000 | |
| polyquaternium-28 | Conditioneze ® NT-20 | 998 | −2 |
| copolymer of isobutylene, | Aquaflex ® FX-64 | 994 | −6 |
| maleimide and | | | |
| hydroxyethylmaleimide | | | |
| polyimide-1 | Aquaflex ® XL-30 | 896 | −104 |
| copolymer of VCL, VP, | Gaffix ® VC-713 | 891 | −109 |
| and DMAEMA | | | |
| polyquaternium-69 | Aquastyle ™ 300 | 887 | −113 |
| copolymer of VP and vinyl | PVP/VA W-735 | 811 | −189 |
| acetate | | | |
| copolymer of VP and | Copolymer 958 | 532 | −468 |
| DMAEMA | | | |
| copolymer of VCL, VP, | Advantage ® LC-A | 373 | −627 |
| and DMAEMA | | | |

TABLE 11B
The effects of various hair styling polymers on viscosity

| | | viscosity | Δviscosity |
|---|---|---|---|
| hair styling polymer | trade name | (cP) | (cP) |
| none | | 29,100 | |
| polyquaternium-11 | Gafquat ® HS-100 | 36,800 | +7,700 |
| copolymer of VP and | Styleze ® CC-10 | 49,700 | +20,600 |
| DMAPA | | | |
| polyquaternium-55 | Styleze ® W-10 | 54,400 | +25,300 |
| copolymer of isobutylene, | Aquaflex ® FX-64 | 52,300 | +23,200 |
| maleimide and | | | |
| hydroxyethylmaleimide | | | |

### Example 11

### Effects of Dioctyl Maleate on Clarity and Viscosity

Compositions were made having 1%, 3%, or 5% (w/w) of lightly- to moderately crosslinked PVP in either C12-C15 alkyl lactates (Ceraphyl® 41) or dioctyl maleate (Ceraphyl®45, ISP). As in the earlier work, turbidity and viscosity were measured as performed in Examples 1 and 2.

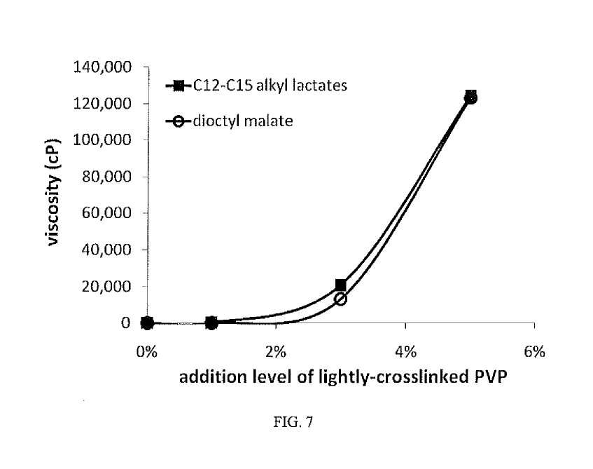

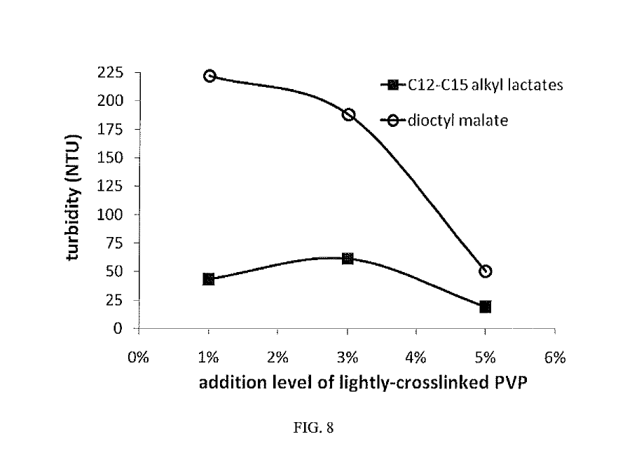

The lightly- to moderately-crosslinked PVP created thickened and clearer oil gels with both Ceraphyl® products. Substantial thickening resulted in both oil with increasing addition level of the polymer (FIG. 7). A 5% addition of the lightly- to moderately-crosslinked PVP promoted viscosities in excess of 120,000 cP. Of course, lesser amounts can be used to achieve lower viscosities. Clearer oil gels also were achieved, notably for the C12-C15 alkyl lactates product (Ceraphyl® 41) (FIG. 8).

### Example 12

### Effects of Dioctyl Maleate on Clarity and Viscosity

Six formulas were created having the phases and ingredients shown in Table 12. The following procedure was followed to prepared them:

1. Heat phase A to 70° C.-75° C., while stirring;

2. Disperse lightly- to moderately-crosslinked PVP into phase A while stirring;

3. Heat phase B to 70° C.-75° C. to dissolve the oil and emulsifier;

4. Add phase A to phase B with homogenizing;

5. Add phase C at 40, stop stirring after mixing well.

Viscosity was measured as for Example 2.

The formulas attained different viscosities due to the lightly- to moderately-crosslinked PVP and glyceryl stearate/laureth-23 (Cerasynth® 945) addition levels. A favorable increase in viscosity occurred when the amount of Cerasynth® 945 was increased, leading to formulations that were stable after centrifuging and storing at 45° C.

TABLE 12
Formulas of Example 12

| | ingredient | | addition level (% w/w) | | | | | |
|---|---|---|---|---|---|---|---|---|
| phase | INCI Name | trade name | a | b | c | d | e | f |
| A | deionized water | | 79.65 | 76.95 | 76.65 | 76.95 | 76.65 | 71.95 |
| | glycerin | | 3.00 | 3.00 | 3.00 | 3.00 | 3.00 | 3.00 |
| | lightly-crosslinked PVP | FlexiThix ™ | 0.00 | 3.00 | 3.00 | 5.00 | 5.00 | 3.00 |
| B | glyceryl stearate & | Cerasynt ® 945 | 0.50 | 0.20 | 0.50 | 0.20 | 0.50 | 0.20 |
| | laureth-23 | | | | | | | |
| | C12-15 alkyl lactate | Ceraphyl ® 41 | 0.00 | 0.00 | 0.00 | 0.00 | 0.00 | 5.00 |
| | isostearyl neopentanoate | Ceraphyl ® 847 | 5.00 | 5.00 | 5.00 | 5.00 | 5.00 | 5.00 |
| | isocetyl Stearoyl stearate | Ceraphyl ® 424 | 4.50 | 4.50 | 4.50 | 4.50 | 4.50 | 4.50 |
| | myristyl myristate & | Ceraphyl ® 424 | 4.50 | 4.50 | 4.50 | 4.50 | 4.50 | 4.50 |
| | myristyl laurate | | | | | | | |
| | refined shea butter | | 2.50 | 2.50 | 2.50 | 2.50 | 2.50 | 2.50 |
| C | PG and DU and IPBC | Liquid Germall ® Plus | 0.35 | 0.35 | 0.35 | 0.35 | 0.35 | 0.35 |

| | | | | | | |
|---|---|---|---|---|---|---|
| total | 100.00 | 100.00 | 100.00 | 100.00 | 100.00 | 100.00 |
| viscosity (cP): | 500 | 20,700 | 36,700 | 73,700 | 127,000 | 4,600 |
| centrifuge at 3000 rpm, 30 min: | separate | separate | stable | stable | stable | separate |
| stability at 45: | separate | stable | stable | stable | stable | stable |

### Example 13

### Effects of Sodium Laureth Sulfate on Clarity and Viscosity

Further examinations were made of sodium laureth-2 sulfate and sodium laureth-3 sulfate and their effect of clarity and viscosity of lightly- to moderately-crosslinked PVP. Each of these surfactants was added at 5%, 10%, and 15% (w/w) to a 5% aqueous preparation of the polymer, Clarity and viscosity were assessed as detailed in Examples 1 and 2.

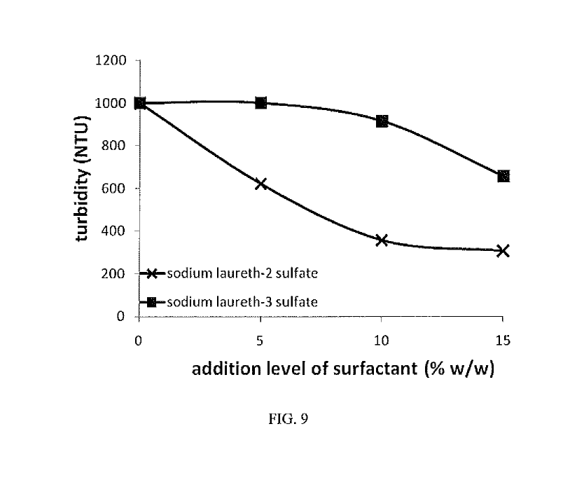

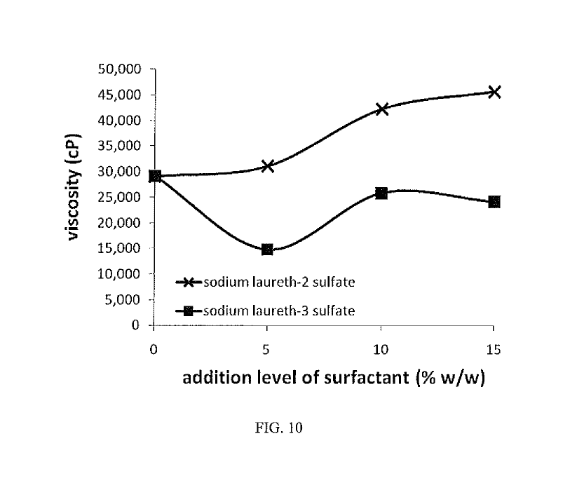

The addition of the surfactants enhanced clarity with increasing addition level, with a greater reduction in turbidity provided by sodium laureth-2 sulfate (FIG. 9). This surfactant also boosted viscosity, whereas sodium laureth-3 sulfate did not (FIG. 10).

### Example 14

### Effects of Ammonium Laureth Sulfate on Clarity and Viscosity

Water-based formulas were created having 5% (w/w) lightly- to moderately-crosslinked PVP and up to 6% (w/w) ammonium laureth sulfate. The method of Example 2 was employed to measure viscosity.

The addition of ammonium laureth sulfate increased the composition viscosity, reaching a maximum of 34,500 cP at the 1.5% addition level (FIG. 11).

What is claimed is:

1. A composition comprising:
(1) at least one additive selected from the group consisting of: cocomidopropyl betaine; decyl glucoside; disodium cocylglutamate; copolymers of isobutylene, maleimide, and hydroxyethylmaleimide; lauramidopropyl betaine; cocamide DEA; decyl glucoside; disodium laureth sulfosuccinate; potassium glycinate; polyquaternium-69; sodium laureth-2 sulfate; sodium laureth-3 sulfate; polyimide-1; copolymers of VP and vinyl acetate; copolymers of VCL, VP, and DMAEMA; potassium lauryl sulfate; PVM/MA decadiene crosspolymer; quaternium-26; sodium cocylglutamate; polyquatemium-55; copolymers of VP, VCL, and DMAPA; potassium cocylglycinate; copolymers of VP and DMAPA; and combinations thereof, and
(2) lightly- to moderately-crosslinked PVP.

2. The composition according to claim 1 wherein said additive is at least 0.5% (w/w) of said composition.

3. The compositions according to claim 1 wherein said additive reduces the turbidity of 5% lightly- to moderately-crosslinked PVP (w/w) by at least 100 NTU compared to a control composition without said additive.

4. The composition according to claim 1 wherein said additive increases the viscosity of 5% lightly- to moderately-crosslinked PVP (w/w) by at least 900 cP compared to a control composition without said additive.

5. A composition consisting of: (A) at least one clarifying additive or at least one viscosifying additive, (B) lightly- to moderately-crosslinked PVP, and (C) at least one solvent.

6. The composition according to claim 5 wherein said clarifying additive is selected from the group consisting of: cocomidopropyl betaine; decyl glucoside; disodium cocylglutamate; copolymers of isobutylene, maleimide, and hydroxyethylmaleimide; lauramidopropyl betaine; potassium glycinate; cocamide DEA; polyquaternium-69; sodium laureth-2 sulfate; sodium laureth-3 sulfate; polyimide-1; copolymers of VP and vinyl acetate; copolymers of VCL, VP, and DMAEMA; potassium lauryl sulfate; potassium cocylglycinate; PVM/MA decadiene crosspolymer; quaternium-26; sodium cocylglutamate; polyquaternium-55; polysorbate-20; ammonium lauryl sulfate; sodium alpha olefin sulfonate; ethanol; sorbitol; sodium lauryl sulfate; butylene glycol; hexylene glycol; copolymers of VP and dimethylaminoethylmethacrylate; copolymers of VP, VCL, and DMAPA; PVP; polyquatemium-11; polyquaternium-28; propylene glycol; glycerin; phenethyl benzoate; and combinations thereof.

7. The composition according to claim 5 having at least 0.5% (w/w) of said clarifying additive.

8. The compositions according to claim 5 wherein said clarifying additive reduces the turbidity of 5% lightly- to moderately-crosslinked PVP (w/w) by at least 100 NTU compared to a control composition without said additive.

9. The composition according to claim 5 wherein said viscosifying additive is selected from the group consisting of: copolymers of VP, VCL, and DMAPA; copolymers of isobutylene, ethylmaleimide, and hydroxyethylmaleimide; polyquatemium-55; copolymers of VP and DMAPA; polyquaternium-28; ammonium lauryl sulfate; sodium lauryl sulfate; sodium laureth-2 sulfate; C12-C₁₅ alkyl lactate; and combinations thereof.

10. The composition according to claim 5 wherein said composition comprises at least 0.5% (w/w) of said viscosifying additive.

11. The composition according to claim 5 wherein said viscosifying additive increases the viscosity of 5% lightly- to moderately-crosslinked PVP (w/w) by at least 900 cP compared to a control composition without said additive.

12. The composition according to claim 5 comprising at least 0.5% (w/w) of said lightly- to moderately-crosslinked PVP.

13. The composition according to claim 5 comprising at least 40% of said solvent.

14. The composition according to claim 5 wherein said solvent is selected from the group consisting of: water, alcohols, esters, glycols, acids, oils, and combinations thereof.

15. The composition according to claim 14 wherein said composition comprises an alcohol selected from the group consisting of: methanol, ethanol, 1-propanol, 2-propanol, 2-methoxypropanol, aminomethyl propanol, 1-butanol, 2-butanol, sec-butanol, 2-aminobutanol, 2-ethylbutanol, 2-methylbutanol, 3-methoxybutanol, behenyl alcohol, amyl alcohol, cetyl alcohol, cinnamyl alcohol, decyl alcohol, hexyl alcohol, cetearyl alcohol, isodecyl alcohol, lauryl alcohol, nonyl alcohol, oleyl alcohol, myristyl alcohol, and combinations thereof.

16. The composition according to claim 14 wherein said composition comprises a glycol selected from the group consisting of: ethylene glycol, propylene glycol, butylene glycol, diethylene glycol, dipropylene glycol, hexylene glycol, hexaethylene glycol, polyethylene glycol, glycerin, and combinations thereof.

17. The composition according to claim 14 wherein said composition comprises an acid selected from the group consisting of: alpha hydroxyethanoic acid, alpha hydroxyoctanoic acid alpha hydroxycaprylic acid, ascorbic acid, adipic acid, citric acid, caprylic acid, capric acid, glycolic acid, lactic acid, lauric acid, malic acid, myristic acid, palmitic acid, salicylic acid, stearic acid, tartaric acid, linoleic acid, linolenic acid, ricinoleic acid, oleic acid, elaidic acid, erucic acid, and mixtures thereof.

18. The composition according to claim 14 wherein said composition comprises an oil selected from the group consisting of: petrolatum, mineral oil, coconut oil, corn oil, cottonseed oil, olive oil, palm oil, peanut oil, rapeseed oil, Canola oil, safflower oil, sesame oil, soybean oil, sunflower oil, almond oil, cashew oil, hazelnut oil, macadamia oil, mongongo oil, pecan oil, pine nut oil, evening primrose oil, blackcurrant seed oil, borage seed oil, and grape seed oil, allspice oil, juniper oil, almond oil, anise oil, celery oil, cumin oil, nutmeg oil, cassia oil, cinnamon oil, sassafras oil, camphor oil, cedar oil, rosewood oil, sandalwood oil, agarwood oil, galangal oil, ginger oil, basil oil, bay leaf oil, common sage oil, eucalyptus oil, lemon grass oil, melaleuca oil, oregano oil, patchouli oil, peppermint oil, pine oil, rosemary oil, spearmint oil, tea tree oil, thyme oil, wintergreen oil, chamomile oil, clary sage oil, clove oil, geranium oil, hops oil, hyssop oil, jasmine oil, lavender oil, manuka oil, marjoram oil, orange oil, rose oil, ylang-ylang oil, bergamot oil, grapefruit oil, lemon oil, tangerine oil, and valerian oil, and combinations thereof.

19. A personal care formulation comprising:
(1) at least one additive selected from the group consisting of cocomidopropyl betaine; decyl glucoside; disodium cocylglutamate; copolymers of isobutylene, maleimide, and hydroxyethylmaleimide; lauramidopropyl betaine; cocamide DEA; decyl glucoside; disodium laureth sulfosuccinate; potassium glycinate; polyquaternium-69; sodium laureth-2 sulfate; sodium laureth-3 sulfate; polyimide-1; copolymers of VP and vinyl acetate;
copolymers of VCL, VP, and DMAEMA; potassium lauryl sulfate; PVM/MA decadiene crosspolymer; quaternium-26; sodium cocylglutamate; polyquaternium-55; copolymers of VP, VCL, and DMAPA; potassium cocylglycinate; copolymers of VP and DMAPA, and
(2) lightly- to moderately-crosslinked PVP.

20. The personal care formulation of claim 19 having the form of a: gel, fluid, spray, cream, lotion, ointment, paste, wax, semi-solid, or solid.

21. The personal care formulation of claim 19 selected from the group consisting of: body wash, skin lotion, sunscreen, anti-wrinkle formula, moisturizer, hair conditioner, anti-perspirant, deodorant, combination anti-perspirant/deodorant, hair rinse, hair shampoo, hair styling agent, mascara, lipstick, lip gloss, and make-up remover.

22. A method of enhancing the clarity, enhancing the viscosity, or enhancing the clarity and the viscosity of a composition comprising lightly- to moderately-crosslinked PVP, said method comprising the steps: (i) selecting a additive selected from the group consisting of cocomidopropyl betaine; decyl glucoside; disodium cocylglutamate; copolymers of isobutylene, maleimide, and hydroxyethylmaleimide; lauramidopropyl betaine; cocamide DEA; decyl glucoside; disodium laureth sulfosuccinate; potassium glycinate; polyquaternium-69; sodium laureth-2 sulfate; sodium laureth-3 sulfate; polyimide-1; copolymers of VP and vinyl acetate; copolymers of VCL, VP, and DMAEMA; potassium lauryl sulfate; PVM/MA decadiene crosspolymer; quaternium-26; sodium cocylglutamate; polyquaternium-55; copolymers of VP, VCL, and DMAPA; potassium cocylglycinate; copolymers of VP and DMAPA and combinations thereof, and (ii) homogenizing said clarifying additive and lightly- to moderately-crosslinked PVP.

23. The method according to claim 22 wherein said additive is at least 0.5% (w/w) of said composition.
